(19) United States
(12) Patent Application Publication
(10) Pub. No.: US 2025/0041089 A1
(43) Pub. Date: Feb. 6, 2025

(54) HEART VALVE PROSTHESIS DELIVERY SYSTEM AND METHOD FOR DELIVERY OF HEART VALVE PROSTHESIS WITH INTRODUCER SHEATH

(71) Applicant: JenaValve Technology, Inc., Irvine, CA (US)

(72) Inventors: Stefan SCHRECK, San Clemente, CA (US); Teodoro S. JIMENEZ, JR., Aliso Viejo, CA (US); Eileen CHARLTON, Garden Grove, CA (US); William GOULD, Santa Fe, NM (US)

(73) Assignee: JenaValve Technology, Inc., Irvine, CA (US)

(21) Appl. No.: 18/920,601

(22) Filed: Oct. 18, 2024

Related U.S. Application Data
(63) Continuation of application No. 15/559,498, filed on Sep. 19, 2017, now Pat. No. 12,121,461.
(60) Provisional application No. 62/136,092, filed on Mar. 20, 2015.

(51) Int. Cl. A61F 2/966; A61F 2/07; A61F 2/24; A61F 2/86; A61F 2/95; A61F 2/958; A61M 25/01; A61M 25/06

(52) U.S. Cl. CPC A61F 2/966; A61F 2/07; A61F 2/2418; A61F 2/2427; A61F 2/2433; A61F 2/2436; A61F 2/86; A61F 2002/9505; A61F 2/9517; A61F 2/9522; A61F 2002/9583; A61F 2250/0006; A61M 25/0136; A61M 2025/0681

(57) ABSTRACT

The present disclosure relates to a delivery system, a catheter system and a method for the minimally invasive application of prostheses to an individual in need thereof and a method for loading a prosthesis onto a catheter system and/or a delivery system.

### CROSS-REFERENCE TO RELATED APPLICATIONS

This application is a continuation of U.S. patent application Ser. No. 15/559,498, filed Sep. 19, 2017, now U.S. Pat. No. 12,121,461, which is a national phase under 35 U.S.C. § 371 of International PCT Patent Application Serial No. PCT/EP2016/055783, filed Mar. 17, 2016, which claims the benefit of U.S. Provisional Patent Application Ser. No. 62/136,092, filed Mar. 20, 2015, the entire contents of each of which are incorporated herein by reference.

### DESCRIPTION

The present disclosure relates to a delivery system, a catheter system and a method for the minimally invasive application of prostheses to an individual in need thereof and a method for loading a prosthesis onto a catheter system and/or a delivery system.

This application is related case of EP13173217.4 & EP13182346.0, U.S. Pat. No. 8,790,395 and US patent publication 2014/0081388A1 (Mar. 20, 2014) all assigned to the instant assignee, the entire disclosures of which are incorporated herein by reference, each as if expressly set forth.

### BACKGROUND

The current disclosure relates to the field of medical devices, in particular to prosthesis which are being transplanted into an individual in need thereof in order to re-establish proper body functions by way of minimally invasive methods and means applicable therefor.

Examples of prostheses that are placed by way of minimally invasive methods are stents and heart valves like aortic and mitral heart valves. Heart valves today are applied e.g. by the transapical, transfemoral, or subclavial route. The transapical route is very direct through a cut between the rips of a patient and through the apex of the heart and thus only a short distance has to be passed until the delivery site. The transfemoral route is a very convenient method and widely used because it needs only a cut in the leg of the patient and access to the vasculature.

Usually the prosthesis is applied by way of a delivery system also denoted catheter. The requirements for the catheter by way of transfemoral delivery is more complex as compared with the transapical route because for the delivery of e.g. an aortic heart valve a relatively long distance and bends have to be manoevered through which imply certain difficulties.

One example of such a delivery system is disclosed in EP2387977B1. This patent describes a transfemoral catheter for the delivery of an aortic heart valve. The patent does not disclose nor suggest the embodiments presently disclosed.

Usually the prosthesis has to be loaded in a releasable manner onto the catheter and crimped to a small seize in order be passed through the vasculature of the patient and to be delivered to the implantation site. The different systems known in the prior art use different seizes like 22, 20, or 18 French. Accordingly the prosthesis has to be squeezed together which implies the risk of damaging the pericard material of the valve.

Moreover, the correct positioning is another issue and possibly the capability of the catheter system of repositioning the prosthesis during the course of delivering and applying the prosthesis at the target site and position in the patient. The termination of the procedure after partial release of the prosthesis and complete retrieval of the prosthesis is another advantage which is not possible with many catheter systems of the prior art.

Another issue is the diameter seize of catheter system. The diameter seize of the crimped prosthesis in the catheter for delivery through the vasculature of the patient is critical. Many known systems do not achieve an adequate crimping seize and often the tissue is negatively affected in known systems during the crimping procedure.

Moreover, the crimping of the prosthesis as such represents a challenge. In particular crimping in a manner without damaging, or stretching and/or squeezing the tissue of the prosthesis is often not sufficiently achieved in known systems. Even though immediate damages due to the crimping may not be obvious, often the tissue is still negatively affected by the crimping with a negative impact on the durability of the tissue, e.g. pericard tissue, representing the valve in a heart valve and even the stent component.

Yet another problem in the delivery is the maneuvering of the prosthesis by way of the catheter through the vasculature and its bends. The fact that the vasculature is narrow, and particularly at the aortic entry into the heart a substantive curve with a narrow angle has to be passed through, represents a substantive challenge for such a delivery procedure and device.

Moreover the following has implications for the present disclosure:

Minimally-invasive surgical procedures have evolved substantially since the advent of endovascular technologies and techniques. Embodiments of the present disclosure relate to systems to deliver an endoprosthesis in an implantation site in the heart of a patient. For example, embodiments of the present disclosure relate to systems to deliver and position transcatheter heart valves used in the treatment of a stenosis (narrowing) of a cardiac valve and/or a cardiac valve insufficiency.

Embodiments of the present disclosure also relate to specialized delivery systems for endoprosthetics, including for example, collapsible and expandable prosthetics incorporating a stent which is delivered to the implant site using a specialized catheter and a preshaped sheath.

The expression “narrowing (stenosis) of a cardiac valve and/or cardiac valve insufficiency” is intended to include a functional defect of one or more cardiac valves, which is either genetic or has developed. A cardiac defect of this type might affect each of the four heart valves, although the valves in the left ventricle (aortic and mitral valves) are affected much more often than the right-sided part of the heart (pulmonary and tricuspid valves). The functional defect can result in narrowing (stenosis), inability to close (insufficiency) or a combination of the two (combined vitium). This disclosure relates to delivery systems leveraging an endoprosthesis that includes an expandable stent capable of being implanted transluminally in a patient's body and enlarged radially after being introduced percutaneously for treating heart valve defect, among other indications.

In the current treatment of severe narrowing of a cardiac valve and/or cardiac valve insufficiency, the narrowed or diseased cardiac valve is replaced with an endoprosthesis. Biological or mechanical valves models, which are typically surgically sewn into the cardiac valve bed through an opening in the chest after removal of the diseased cardiac valve, have generally been used for this purpose. Such procedures or operations necessitate the use of a heart-lung machine to maintain the patient's circulation during the procedure and cardiac arrest is induced during implantation of the prosthesis. This is a risky surgical procedure with associated dangers for the patient, as well as a long postoperative treatment and recovery phase. Such an operation can often not be considered with justifiable risk in the case of polypathic patients.

Minimally-invasive forms of treatment have been developed recently which are characterized by allowing the procedure to be performed under local anesthesia. One approach provides for the use of a catheter system to implant a self-expandable stent to which is connected a collapsible valvular prosthesis. Such a self-expandable endoprosthesis can be guided via a catheter system to the implantation site within the heart through an inguinal artery or vein. After reaching the implantation site, the stent can then be unfolded.

However, there is a risk of inexact or incorrect implantation of an endoprosthesis using the solutions described above. Expressed in another way, there is a need for exact positioning and longitudinal alignment of an implanted endoprosthesis. In particular, it is only possible using great skill on the part of the attending surgeon or cardiologist-if at all-to position a stent sufficiently precisely, in both a lateral and longitudinal direction, to ensure that the associated endoprosthesis is located in the correct area of the patient's diseased heart valve to be treated prior to the advent of the instant systems.

On the basis of the problems referenced and outlined above, certain embodiments of the present disclosure address the issue of delivering and positioning a specialized endoprosthesis for treating a narrowed cardiac valve or a cardiac valve insufficiency which realizes optimum positioning accuracy and anchoring of the emplaced device. In addition, the treatment of the narrowed cardiac valve or cardiac valve insufficiency should be by way of a simple procedure to enable routine treatment of narrowed cardiac valve or cardiac valve insufficiency without major stress to the patient.

### Objects of the Disclosure

One object of certain embodiments of the present disclosure was to provide a catheter system for delivery of a prosthesis, e.g. a heart valve, which is easy to handle. In particular wherein the heart valve can securely be loaded and crimped without negative side effects due to the loading and crimping procedure.

Another object of certain embodiments of the present disclosure is to provide for a catheter and delivery system for a prosthesis designed in a manner in order to facilitate the delivery of the prosthesis to the target site. In particular provide for a system design wherein the maneuvering through the vasculature of a patient is possible without the disadvantages known in the prior art.

Another object of certain embodiments of the present disclosure is to provide for a design wherein the prosthesis can be securely positioned and delivered to the target site with the possibility to terminate the delivery procedure and to retract the prosthesis until a certain procedural time point. Moreover, it is an object of certain embodiments of the present disclosure to enable the free movement of the prosthesis in axial direction and rotational direction.

Another object of certain embodiments of the present disclosure is to provide for a step-wise liberation of the prosthesis in order to place the prosthesis correctly at the target site, enable repositioning in this manner and/or fine tuning of the positioning procedure.

### SUMMARY

Embodiments of the present disclosure relate in one aspect to a catheter system comprising i. a steering means; and ii. a delivery means for a heart valve, optionally balloon expandable or self-expandable, wherein the steering means and the delivery means are coaxially and rotationally independently movable.

In another aspect, embodiments of the present disclosure relate to a delivery system for a heart valve comprising i. a steering means; ii. a delivery means for a heart valve and iii. an introducer sheath wherein the steering means, the delivery means and the introducer sheath are coaxially and circumferentially independently movable.

In yet another aspect, embodiments of the present disclosure relate to a method for the delivery of a heart valve to the heart of a patient comprising the steps i. in a first step placing an introducer comprising an introducer sheath into a patient's vasculature; ii. in a second step introducing a catheter system having fixed thereto a self-expanding heart valve through the introducer into the patient's vasculature; iii. in a third step positioning the self-expanding heart valve proximal to the target site in the patient's heart, optionally positioning the heart valve substantially central in the target area; iv. in a fourth step positioning the self-expanding heart valve at the target site; v. in a fifth step partially releasing the self-expanding heart valve from the catheter system; vi. in a sixth step fully releasing the self-expanding heart valve from the catheter system.

In yet another aspect, embodiments of the present disclosure relate to a method of loading a self-expanding heart valve onto a delivery means of a catheter system having an inner shaft, an attachable distal tip, and at least one outer sheath, consisting of compressing the self-expanding heart valve radially and placing the compressed self-expanding heart valve into a tubular retainer, the retainer preventing the self-expanding valve from expanding radially, passing the inner shaft of the delivery system through the central opening of the compressed self-expanding heart valve, attaching the distal tip to the shaft of the delivery system, advancing the outer sheath onto the compressed heart valve to prevent radial expansion of at least part of the heart valve, and removing the tubular retainer from the compressed self-expanding heart valve.

In yet another aspect, embodiments of the present disclosure relate to a 2-step deployment procedure of a heart valve.

In yet another aspect, embodiments of the present disclosure relate in particular to the attachment of a heart valve in a catheter or delivery system wherein only the lateral parts of the heart valve are fixed and/or covered by a fixation means of the catheter and wherein essentially the middle part of the heart valve is not being covered.

In yet another aspect, embodiments of the present disclosure relate to a steerable catheter for the delivery of a heart valve.

Briefly stated, transcatheter heart valves are delivered by transapical (TA) and/or transfemoral (TF) delivery systems featuring novel preshaped sheaths and/or steerable catheters to enable emplacement leveraging benefits of proprietary JenaValve® brand of prosthetics.

According to the some embodiment, transcatheter heart valve delivery systems include a Pericardial THV system which comprises a specialized prosthesis; a prosthetic delivery system and separate valve loader.

According to some embodiments, the prosthesis is constructed from porcine pericardial tissue and attached to a self-expanding Nitinol stent scaffold with polyester sutures, having specialized struts and the ability to fit into either transapical or transfemoral catheter delivering devices, as further described, illustrated and claimed below.

According to some embodiments, transapical and/or transfemoral catheter delivery systems are effective to collapsibly house and deliver the scaffold, which has a crown-like design, and works with a pre-shaped sheath and/or steerable catheters, as further described, illustrated and claimed below.

According to some embodiments, the instant systems match with at least three valve sizes, accommodating native valve annulus diameters from at least about 21 to 27 mm.

In accordance with an aspect, the present disclosure provides a catheter system for introducing an expandable heart valve into the body of a patient, the catheter system comprising delivering means for the heart valve.

In some aspects of the present disclosure, the delivering means comprises a catheter tip and a catheter shaft. The catheter tip of the delivery means has a seat portion for accommodating the heart valve to be introduced into the patient's body in its collapsed or crimped state. The catheter tip has further holding means for realisably fixing the heart valve to the catheter tip.

The seat portion of the catheter tip is constituted by a first retaining means and a second retaining means. In some embodiments of the present disclosure, the first retaining means and second retaining means may be constituted by a first sleeve-shaped member and a second sleeve-shaped member.

The first retaining means of the catheter tip serves for reversibly securing a distal end section of the heart valve to the delivery means and, in particular, to the catheter tip of the delivery means. On the other hand, the second retaining means of the catheter tip serves for reversibly securing a proximal end section of the heart valve to the delivery means and, in particular, to the catheter tip of the delivery means.

The first and second retaining means are moveable relative to each other and also relative to the holding means of the catheter tip.

According to some embodiments of the present disclosure, the catheter shaft of the delivery means comprises first force transmitting means and second force transmitting means.

A distal end section of the first force transmitting means is connected or connectable to the first retaining means of the catheter tip and a proximal end section of the first force transmitting means is connected or connectable to a first operating means of a handle of the delivery means. A distal end section of the second force transmitting means is connected or connectable to the second retaining means of the catheter tip and a proximal end section of the second force transmitting means is connected or connectable to a second operating means of the handle of the delivery means.

According to some embodiments of the present disclosure, the handle of the delivery means has at least one first operating means and at least one second operating means with which the catheter tip of the delivery means may be appropriately manipulated so that an expandable heart valve secured to the catheter tip may be released from the catheter tip in steps or in a defined or definable sequence of events.

In accordance with preferred embodiments of the present disclosure, the catheter tip has first and second retaining means—for example in the form of sleeve-shaped members which may be manipulated with the handle of the delivery means. These retaining means are used for releasably and reversibly securing a distal and proximal end section of the heart valve to the catheter tip.

In some embodiments of the present disclosure, the first retaining means serves for releasably and reversibly securing first functional components of the heart valve, for example retaining hoops of a stent or alternatively positioning hoops of a stent, while the second retaining means serves for releasably and reversibly securing second functional components of the heart valve, for example, positioning hoops of a stent or alternatively for accommodating retaining hoops of a stent.

In relation to the handle of the delivery means, it is preferably provided that, on one hand, the first operating means cooperate with the first retaining means of the catheter tip so that, on actuation of the first operating means, a previously definable longitudinal displacement of the first retaining means may be effected relative to the holding means. On the other hand, the second operating means cooperates with the second retaining means of the catheter tip so that a previously definable longitudinal displacement of the second retaining means may be affected relative to the holding means.

In accordance with some embodiments of the present disclosure, the first and second retaining means only serve for securing the distal and proximal end sections of the heart valve to the catheter tip. In case the first and second retaining means are configured as first and second sleeve-shaped members, these sleeve-shaped members have a length such that a gap is between the first and second sleeve-shaped members when securing the distal and proximal end sections of the heart valve to the catheter tip.

In accordance with some embodiments of the present disclosure, the first force transmitting means of the delivery means is constituted by a first catheter tube defining a first lumen, and the second force transmitting means of the delivery means is constituted by a second catheter tube defining a second lumen. The second catheter tube has a cross-section less than the cross-section of the first catheter tube. The first catheter tube is disposed concentrically and coaxially with the second catheter tube and the second catheter tube is received within the first lumen defined by the first catheter tube.

Contrary to the first and second sleeve-shaped members of the catheter tip, however, the holding means of the catheter tip is not moveable relative to the handle of the delivery means. Rather, the holding means is connected to the handle of the delivery means by using a holding tube having a distal end connected to the holding means and a proximal end connected to a body of the handle of the delivery means. The holding tube has a cross-section less than the cross-section of the first catheter tube. In particular, the first catheter tube is disposed concentrically and coaxially with both, the second catheter tube on the one hand and the holding tube on the other hand.

Preferably, the holding tube has a cross-section less than the cross-section of the first catheter tube and greater than the cross-section of the second catheter tube such that the holding tube is received within the first lumen defined by the first catheter tube and the second catheter tube is received within a passageway defined by the holding tube. The passageway defined by the holding tube has a diameter sufficient to accommodate the second catheter tube such that the second catheter tube is moveable relative to the holding tube.

The second lumen defined by the second catheter tube has a diameter sufficient to accommodate a guide wire. The second catheter tube is made from a rigid material including, for example, nitinol, stainless steel or a rigid plastic material. The material of the distal end section of the second catheter tube may have an increased flexibility compared to the material of the proximal end section in order to allow the distal end section of the catheter shaft to pass the aortic arch during insertion of the catheter tip.

In some embodiments of the present disclosure, the distal end section of the second catheter tube terminates in a soft catheter end tip having an atraumatic shape. The soft catheter end tip is provided with a channel aligned with the second lumen defined by the second catheter tube such that a guide wire accommodated within the second lumen of the second catheter tube may pass through the channel of the soft catheter end tip. The second sleeve-shaped member of the catheter tip is connected to the soft catheter end tip such that the opened end of the second sleeve-shaped member faces in the proximal direction opposite to the direction of the soft catheter end tip and to the second catheter tube.

The holding tube is preferably made of a rigid material, for example, a rigid plastic material, stainless steel or Nitinol. The distal end of the holding tube terminates in the holding means which is also made of a rigid material, for example, a rigid plastic material or stainless steel. The passageway defined by the holding tube is aligned with a channel which passes through the holding means. In this way, the second catheter tube is accommodated in the passageway of the holding tube and the channel of the holding means such as to be moveable relative to the holding tube and the holding means.

The holding tube is provided for connecting the holding means to the handle of the delivery means. For this purpose, the holding tube has a distal end connected to the holding means and a proximal end connected to a body of the handle of the delivery means.

The first catheter tube is preferably made of a bendable but inelastic material. For example, the first catheter tube may be at least partly made of a braided or non-braided catheter tube. Hence, the first catheter tube may have a stiff braid reinforced body similar to the catheter body described in U.S. Pat. No. 4,665,604 which is incorporated herein by reference.

The first catheter tube shall be adapted to transfer compression and tension forces from the first operating means of the handle of the delivery means to the first retaining means of the catheter tip without overly changing of its total length. The distal end of the first catheter tube terminates at a flared section as the transition to the section defining the first retaining means of the catheter tip.

The flared section and the first retaining means may be formed integrally and may be connected to the distal end section of the first catheter tube. Alternatively, the first retaining means and the flared section of the first catheter tube may be all of the same material and originating from the same raw tube prior to a widening process so that the flared section and the first retaining means are the same elements.

In accordance with another aspect of the present disclosure, the catheter system further comprises steering means. The steering means serves for guiding the delivery means and, in particular, the catheter tip of the delivery means when advancing the catheter tip through the patient's vasculature.

In some embodiments of the present disclosure, the steering means comprises a steerable catheter tube having a proximal end section and a distal end section. The proximal end section of the steerable catheter tube is connected or connectable with a handle of the steering means.

The handle of the steering means is provided with operating means, for example in the form of a rotatable knob or wheel, by means of which a flexural link region of the steerable catheter tube can be controlled.

In accordance with other embodiments disclosed herein, the steerable catheter tube of the steering means is provided without any flexural link region, which can be manipulated by corresponding operating means. Rather, in these embodiments, the material of the distal end section of the steerable catheter tube may have an increased flexibility compared to the material of the proximal end section. In this way, the distal end section of the steerable catheter tube may easily pass, for example, the aortic arch during insertion of the steerable catheter tube.

In some embodiments disclosed herein, the handle of the steering means is provided with operating means by means of which a flexural link region of the steerable catheter tube can be controlled, wherein the operating means preferably has a detent device to allow a set deflection of the flexural link region of the steerable catheter tube to be fixed. For example, it is possible to provide a suitable catch mechanism on the operating means, which cooperates with a body of the handle of the steering means. In particular, it is possible for the flexural link region of the steerable catheter tube to be connected to the operating means of the steering means by way of a control wire whereby, on an actuation of the operating means via the control wire a tensile forces is exerted on the flexural link region of the steerable catheter tube, which produces a predefined or predefinable deflection of the flexural link region.

However it is also possible, of course, to choose other embodiments as the operating means of the steering means for deflecting the steerable catheter tube or a flexural link region of the steerable catheter tube, in case the steerable catheter tube is provided with such a flexural link region.

In accordance with some embodiments disclosed herein, the proximal end section of the steerable catheter tube of the steering means terminates in a port section of the steering means or is connected with a port section of the steering means. The port section of the steering means serves for introducing the catheter tip and catheter shaft of the delivery means into the steerable catheter tube. For this purpose, the port section of the steering means has a lumen defining a passageway which extends through the port section, the distal end section of the passageway being aligned with the proximal end section of the steerable catheter tube.

The port section of the steering means is preferably integrated in or connected with the handle of the steering means.

The catheter tip and catheter shaft of the delivery means is introducible (via the port section of the steering means) into the steerable catheter tube. The catheter shaft of the delivery means and particularly the first catheter tube of the catheter shaft of the delivery means is moveable relative to the steerable catheter tube. In particular, the steerable catheter tube terminates proximal to the catheter tip wherein the cross-section of proximal end section of the introducer sheath shall be substantially the same as or slightly larger than the cross-section of the flared section provided at the proximal end of the first catheter tube.

The proximal end section of the steering means and, in particular, the proximal end section of the port section of the steering means is releasably connectable to the handle of the delivery means.

According to an aspect of the present disclosure, the catheter system further comprises an introducer having an introducer sheath. The introducer sheath has a cross-section greater than the cross-section of the steerable catheter tube of the steering means.

The introducer sheath serves as guiding means when introducing the delivery means and/or the steering means and, in particular, the steerable catheter tube of the steering means into the patient's vasculature. In more detail, the introducer sheath defines a passageway through which the catheter tip and catheter shaft of the delivery means and/or the steerable catheter tube of the steering means may be guided to the implantation side within the patient's body.

The introducer sheath has a distal end, a proximal end and a passageway extending there between. The introducer sheath has a length such that the distal end of the introducer sheath terminates proximal to the catheter tip of the delivery system, when the catheter shaft and the catheter tip of the delivery system has been fully introduced into the introducer sheath.

When at least a section of the catheter shaft of the delivery means or at least a section of the steerable catheter tube of the steering means has been introduced into the passageway defined by the introducer sheath, the introducer sheath is disposed concentrically and coaxially with the section of the catheter shaft of the delivery means or the steerable catheter tube of the steering means.

In any case, however, the catheter shaft of the delivery means and/or the steerable catheter tube of the steering means is moveable relative to the introducer sheath. In particular, the introducer sheath terminates proximal to the catheter tip wherein the cross-section of proximal end section of the introducer sheath shall be substantially the same as or slightly larger than the cross-section of the steerable catheter tube.

The proximal end section of the introducer sheath is connected to an introducer port. The introducer port serves for providing access to the introducer sheath of the introducer required for delivering the delivery means or the steerable catheter tube of the steering means into the introducer sheath.

The introducer port may comprise a base member which is configured to be manually fixable to the handle of the delivery means, when the catheter shaft of the delivery means is introduced into the passageway defined by the introducer sheath, or to be manually fixable to the handle of the steering means, when the steerable catheter tube of the steering means is introduced into the passageway defined by the introducer sheath.

In accordance with some embodiments disclosed herein, the introducer port is provided with a sealing arrangement for preventing leakage of fluid, in particular blood, from the introducer, when the introducer sheath is introduced into the patient's vasculature.

In some embodiments disclosed herein, the proximal end section of the introducer sheath terminates in a crimping section distal to the sealing arrangement of the introducer port. The crimping section of the introducer serves for crimping at least a middle section of a heart valve fixed to the catheter tip of the delivery means during the introduction of the catheter tip into the introducer sheath. As already mentioned above, according to embodiments disclosed herein, preferably only the distal and proximal end sections of the heart valve are fixed to the catheter tip of the delivery means by means of the first and second retaining means, wherein no dedicated retaining means is allocated to the middle section of the heart valve between the distal and proximal end sections of the heart valve.

For crimping the middle section of the heart valve during the introduction of the catheter tip into the introducer sheath, the catheter tip passes through the crimping section of the introducer thereby further reducing the diameter of the middle section of the heart valve fixed to the catheter tip. In some embodiments disclosed herein, the crimping section may comprise a conical tubular member having an inner diameter which decreases in the distal direction of the tubular member.

The introducer sheath may be of a thin material such as to allow length deformation of the introducer sheath upon transfer of compression and tension forces. The introducer sheath material, however, shall have sufficient stiffness in order to mechanically avoid kinking of the flexible sections of the distal portion of the catheter shaft during insertion of the catheter tip.

In some embodiments of the present disclosure, the introducer sheath has a pre-shaped, preferably curved, configuration.

An inlet may be provided at a proximal end section of the steering means, the introducer and/or the delivery means for injection of fluids, if necessary. Furthermore, a check valve may be provided at the proximal end section of the introducer sheath to prevent fluid from leaking out of the introducer sheath.

The introducer sheath may have a length sufficient to protect the inner wall of the blood vessel through which the catheter tip passes. In addition, a separate introducer system (not belonging to the catheter system) may be provided. The introducer system then may serve as a portal for passing the complete catheter system from the catheter tip to the catheter shaft into the patient's body and up to the heart.

In addition, the introducer sheath reduces the compression force exerted on the first catheter tube that is inserted through the introducer sheath. This increases manoeuvrability of the steerable catheter tube and the catheter shaft of the delivery means throughout the procedure. A consequence thereof is that any frictional force is reduced. Moreover, moving the catheter tip after it has been advanced through the vascular system of a patient, is greatly improved while at the same time lowering the risk of injury of the patient.

The length of the introducer sheath depends on the length of the catheter shaft of the delivery means and will typically be between about 20 cm and 100 cm. Those skilled in the art will appreciate, however, that all dimensions provided herein are intended as examples only, and that the introducer sheaths and catheter tubes of different dimensions may be substituted for a particular use.

As will be appreciated, the introducer sheath will be of a size, i.e. has an outer diameter, which will permit insertion in a patient's blood vessel (artery or vein) which is used for moving the stent transarterially or via a vein to an insufficient heart valve.

The introducer sheath may be capable of traversing tortuous pathways in the body of the patient without kinking. The introducer sheath may include an inner lubricious liner, an outer polymeric jacket, and a coil reinforcement between the inner and the outer layers. This introducer sheath may provide favourable flexibility without kinking or compression. One or more radiopaque bands or markers may be incorporated within the introducer sheaths material to allow precise location of the introducer sheaths distal end for positioning accuracy. Those skilled in the art will appreciate that other known materials may also be suitable for a particular purpose.

The catheter system is particularly adapted to deliver and implant a heart valve as described for example in the European Patent Application No. 07 110 318 or in the European Patent Application No. 08 151 963. In some embodiments of the present disclosure, a heart valve is accordingly used which comprises a stent and a heart valve prosthesis attached to the stent. The stent exhibits the following:
a first retaining region, to which the heart valve prosthesis can be attached;
an opposing, second retaining region with at least one retaining element, for example in the form of retaining eyes or in the form of retaining heads, whereby at least one retaining element of the stent can be put in releasable engagement with the holding means of the catheter tip of the delivery means;
at least one retaining hoop, to which the heart valve prosthesis can be fastened; and
at least one and preferably three positioning hoops, which are designed to engage in pockets of the native heart valve in the implanted state of the stent, thus to enable automatic positioning of the stent in the aorta of the patient.

In particular, a catheter system is disclosed herein, with which an expandable heart valve stent with a heart valve prosthesis attached to this stent can be advanced to the implantation site in a particularly simple way, for example via the aorta of a patient being treated (transarterially or transfemorally). Preferably, during transarterial or transfemoral access by the catheter system, the whole free cross-section available within the aorta is not completely filled up, since the catheter tip provided at the distal end region of the catheter system, in which the stent can be accommodated with the heart valve prosthesis, can be made sufficiently small with respect to its external diameter.

The expandable heart valve stent with the heart valve prosthesis attached to it can be fixed temporarily during implantation in a crimped state to the catheter tip of the delivery means.

The catheter system designed for transarterial or transfemoral access is therefore suitable for inserting a heart valve stent with a heart valve prosthesis attached to it, transarterially or transfemorally into the body of the patient; for example, the catheter tip of the delivery means of the catheter system is inserted via puncture of the A. femoris communis (inguinal artery).

In particular, with the catheter system designed for transarterial or transfemoral access, the catheter shaft of the delivery means may be designed so that it is both kink-resistant and flexible such that a bending radius of up to 4 cm, and preferably up to 3 cm, can be realised, at least at the distal end region of the catheter shaft.

### BRIEF DESCRIPTION OF THE DRAWINGS

Various embodiments will be described with reference to the appended drawings below. Of these:

FIG. 1: a side elevation of an embodiment of a delivery system for transfemoral/transarterial insertion of an expandable heart valve;

FIG. 2a: a side elevation of an embodiment of a delivery means of a delivery system in accordance with FIG. 1;

FIG. 2b: a sectioned side elevation of the delivery means of FIG. 2a;

FIG. 2c: a sectioned side elevation of the catheter tip of the delivery means of FIG. 2a;

FIG. 3a: a side elevation an embodiment of a steering means of a delivery system in accordance with FIG. 1;

FIG. 3b: a sectioned side elevation of the steering means of FIG. 3a;

FIG. 4: a sectioned side elevation of an embodiment of an introducer of a delivery system in accordance with FIG. 1;

FIG. 5: a schematic view to illustrate a transfemoral/transarterial implantation procedure of a heart valve stent;

FIGS. 6a-i: sectioned side elevations of components of the delivery means of FIG. 2a;

FIG. 7: a plan view of a full system for TF transcatheter heart valve delivery, according to the instant disclosure;

FIG. 8: a close up of the system of FIG. 1, according to the instant disclosure;

FIG. 9: a schematic of the JenaValve® brand of prosthetic, and an actual view of the same;

FIG. 10: a depiction of the TF delivery system;

FIG. 11: another view of the TF delivery system;

FIG. 12: an additional view illustrating a pre-shaped sheath, according to the instant disclosure;

FIGS. 13a, b: side views of an embodiment of a device to facilitate the removal of the distal end of a catheter from an introducer sheath or guiding catheter;

FIGS. 14a, b: side views of another embodiment of a device to facilitate the removal of the distal end of a catheter from an introducer sheath or guiding catheter;

FIG. 15: a side view of another embodiment of a device to facilitate the removal of the distal end of a catheter from an introducer sheath or guiding catheter;

FIGS. 16a, b: side views of another embodiment of a device to facilitate the removal of the distal end of a catheter from an introducer sheath or guiding catheter;

FIG. 17: a side view of another embodiment of a device to facilitate the removal of the distal end of a catheter from an introducer sheath or guiding catheter;

FIGS. 18a, b: side views of another embodiment of a device to facilitate the removal of the distal end of a catheter from an introducer sheath or guiding catheter;

FIGS. 19a, b: side views of another embodiment of a device to facilitate the removal of the distal end of a catheter from an introducer sheath or guiding catheter; and

FIG. 20: a side view of another embodiment of a device to facilitate the removal of the distal end of a catheter from an introducer sheath or guiding catheter.

### DETAILED DESCRIPTION

In the following, exemplary embodiments of the present disclosure will be described in more detail.

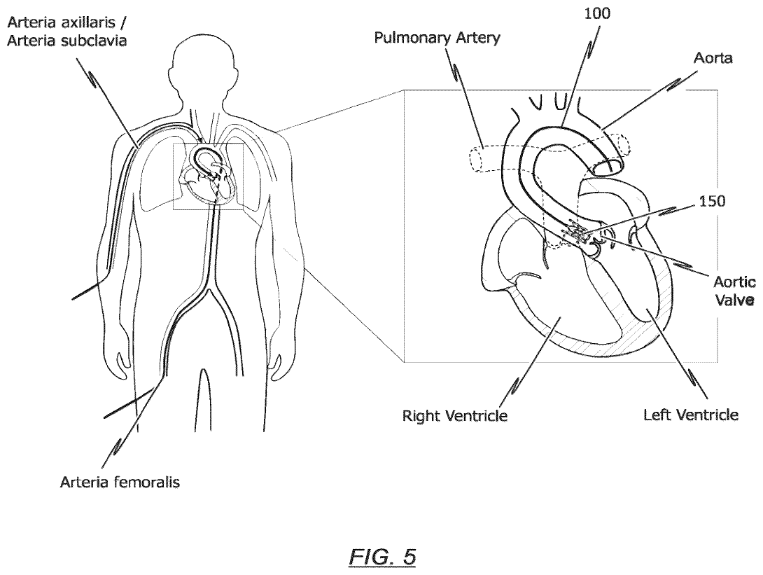

FIG. 5 shows schematically an example of how a transarterial or transfemoral access can be gained to the heart of a patient. In the illustration in accordance with FIG. 5, a heart valve stent 150 is advanced with the aid of a delivery system 100 via the femoral artery to the aortic valve. Embodiments of a delivery system 100, which is suitable for transarterial or transfemoral access, are described in the following.

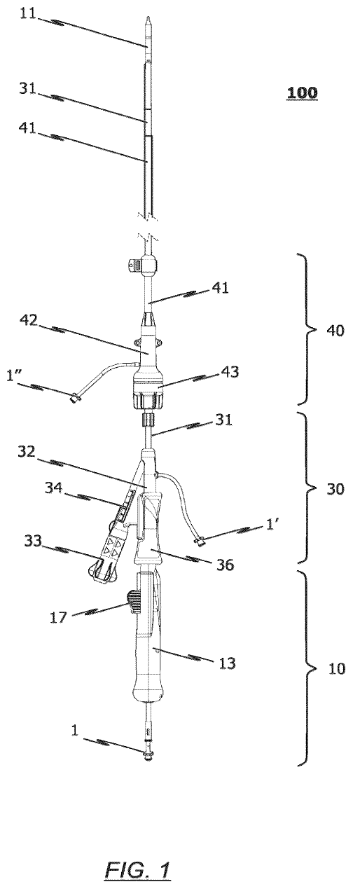

As depicted, for example, in FIG. 1, the delivery system 100 according to an exemplary embodiment of the present disclosure comprises delivery means 10, steering means 30, and an introducer 40.

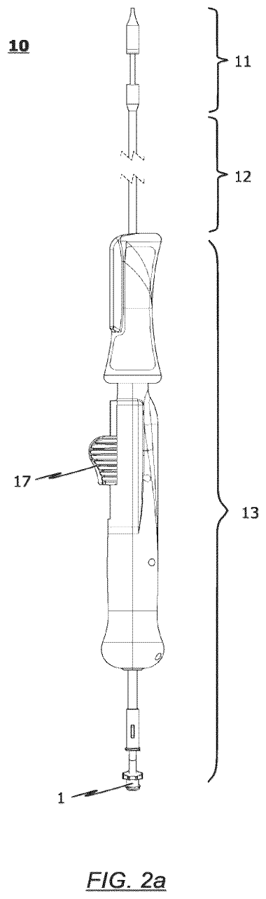

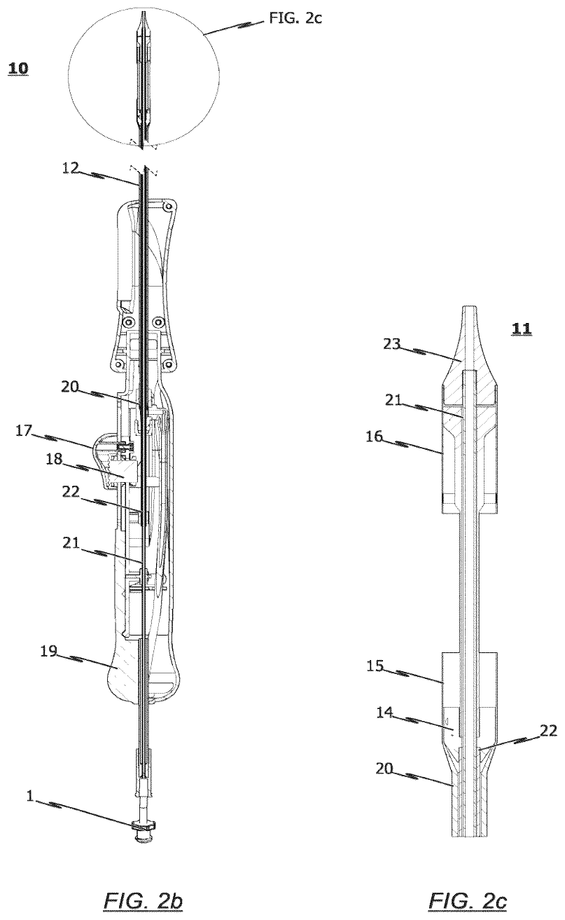

The delivery means 10 has a catheter tip 11, a catheter shaft 12, and a handle 13 connected to the proximal end section of the catheter shaft 12. In this regard, reference is also made to FIG. 2a and FIG. 2b.

As illustrated, for example, in FIG. 2c, the catheter tip 11 of the delivery means 10 has a seat portion for accommodating a heart valve to be inserted in its crimped state and holding means 14 for releasably fixing the heart valve to the catheter tip 11.

The delivery means 10 further comprises a catheter shaft 12 for connecting the catheter tip 11 to the handle 13 of the delivery means 10, the distal end section of the catheter shaft 12 being flexible enough such that the catheter tip 11 and the distal end section of the catheter shaft 12 may pass the aortic arch during insertion through the aorta of the patient.

The seat portion of the catheter tip 11 comprises first retaining means 15 and second retaining means 16, the respective cross-sections of the first and second retaining means 15, 16 may be identical to each other. In the exemplary embodiment illustrated in FIG. 2c, the first and second retaining means 15, 16 are configured as sleeve-shaped elements, respectively. The lengths of these sleeve-shaped elements are respectively chosen such that the seat portion of the catheter tip is not totally covered by the sleeve-shaped elements. Rather and as illustrated in FIG. 2c, a middle section of the seat portion of the catheter tip is always uncovered.

The first and second retaining means 15, 16 are movable relative to each other and relative to the holding means 14. For this purpose, first force transmitting means 20 with a distal end section connected to the first retaining means 15 and a proximal end section connected to first operating means 17 of the handle 13 are provided. In addition, second force transmitting means 21 with a distal end section connected to the second retaining means 16 and a proximal end section connected to second operating means 18 of the handle 13 are provided. When manipulating the first and/or second operating means 17, 18 of the handle 13, the first and/or second retaining means 15, 16 may be moved relative to each other and relative to the holding means 14.

As can be seen from FIG. 2b and FIG. 2c, the first force transmitting means 20 may be constituted by a first catheter tube defining a first lumen and the second force transmitting means 21 is constituted by a second catheter tube defining a second lumen. The second catheter tube may have a cross-section less than the cross-section of the first catheter tube. The first catheter tube may be disposed concentrically and coaxially with the second catheter tube and the second catheter tube is received within the first lumen defined by the first catheter tube.

Contrary to the first and second retaining means 15, 16 of the catheter tip 11, however, the holding means 14 of the catheter tip 11 are not moveable relative to the handle 13 of the delivery means 10. Rather, the holding means 14 are connected to the body 19 of the handle 13 by using a holding tube 22 having a distal end connected to the holding means 14 and a proximal end connected to a body 19 of the handle 13.

The holding tube 22 may have a cross-section less than the cross-section of the first catheter tube of the first force transmitting means 20. In particular, the first catheter tube of the first force transmitting means 20 may be disposed concentrically and coaxially with both, the second catheter tube of the second force transmitting means 21 on the one hand and the holding tube 22 on the other hand.

In some embodiments, the holding tube 22 has a cross-section less than the cross-section of the first catheter tube and greater than the cross-section of the second catheter tube such that the holding tube 22 is received within the first lumen defined by the first catheter tube and the second catheter tube is received within a passageway defined by the holding tube 22. The passageway defined by the holding tube 22 has a diameter sufficient to accommodate the second catheter tube such that the second catheter tube is moveable relative to the holding tube 22.

The second lumen defined by the second catheter tube has a diameter sufficient to accommodate a guide wire (not shown). The second catheter tube may be made from a rigid material including, for example, Nitinol, stainless steel or a rigid plastic material. The material of the distal end section of the second catheter tube may have an increased flexibility compared to the material of the proximal end section in order to allow the distal end section of the catheter shaft 12 to pass the aortic arch during insertion of the catheter tip 11.

As can been seen, for example, from FIG. 2c, the distal end section of the second catheter tube terminates in a soft catheter end tip 23 having an atraumatic shape. The soft catheter end tip 23 is provided with a channel aligned with the second lumen defined by the second catheter tube such that a guide wire accommodated within the second lumen of the second catheter tube may pass through the channel of the soft catheter end tip. The second retaining means 16 of the catheter tip 11 is connected to the soft catheter end tip 23 such that the opened end of the second retaining means 16 faces in the proximal direction opposite to the direction of the soft catheter end tip 23 and to the second catheter tube.

According to the exemplary embodiments of the present disclosure, the holding tube 22 is made of a rigid material, for example, a rigid plastic material, stainless steel or Nitinol. The distal end of the holding tube 22 terminates in the holding means 14 which is also made of a rigid material, for example, a rigid plastic material or stainless steel. The passageway defined by the holding tube 22 is aligned with a channel which passes through the holding means 14. In this way, the second catheter tube is accommodated in the passageway of the holding tube 22 and the channel of the holding means 14 such as to be moveable relative to the holding tube 22 and the holding means 14.

The first catheter tube of the first force transmitting means 20 is made of a bendable but inelastic material. For example, the first catheter tube may be at least partly made of a braided or non-braided catheter tube. The first catheter tube shall be adapted to transfer compression and tension forces from the first operating means 17 of the handle 13 to the first retaining means 15 of the catheter tip 11 without overly changing its total length. The distal end of the first catheter tube terminates at a flared section as a transition to the section defining the first retaining means 15 of the catheter tip 11.

The flared section and the first retaining means 15 may be formed integrally and may be connected to the distal end section of the first catheter tube. In addition, the flared section may constitute the first retaining means 15 of the catheter tip 11. The first retaining means 15 and the flared section of the first catheter tube may be all of the same material and originating from the same raw tube prior to a widening process so that the flared section and the first retaining means 15 are the same elements.

Referring for example to FIG. 1, the delivery system 100 according to an exemplary embodiment of the present disclosure further comprises steering means 30 for guiding the delivery means 10 and, in particular, the catheter tip 11 of the delivery means 10 when advancing the catheter tip 11 through the patient's vasculature.

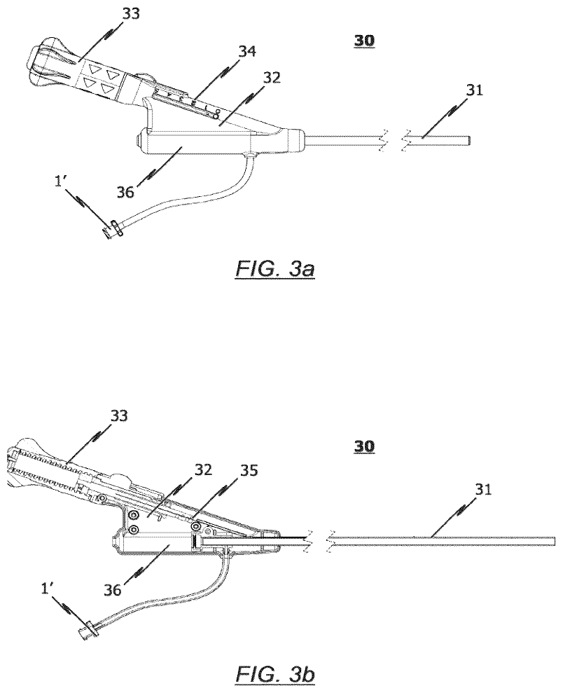

As illustrated in FIGS. 3a-b, the steering means 30 comprises a steerable catheter tube 31 having a proximal end section and a distal end section. The proximal end section of the steerable catheter tube 31 is connected or connectable with a handle 32 of the steering means 30.

The handle 32 of the steering means 30 is provided with operating means 33, for example in the form of a rotatable knob or wheel, by means of which a flexural link region of the steerable catheter tube 31 can be controlled.

As illustrated in FIG. 3a, the handle 32 of the steering means 30 is also provided with indicator means 34 for indicating the degree of deflection applied to the flexural link region of the steerable catheter tube 31 by means of the operating means 33.

Although not explicitly shown in FIG. 3a, in some embodiments, the operating means 33 preferably has a detent device to allow a set deflection of the flexural link region of the steerable catheter tube 31 to be fixed. For example, it is possible to provide a suitable catch mechanism on the operating means 33, which cooperates with a body of the handle 32 of the steering means 30.

As can be seen from FIG. 3b, a control wire 35 is provided, which connects the operating means 34 with the flexural link region of the steerable catheter tube 31. On an actuation of the operating means 33, a tensile forces is exerted via the control wire 35 on the flexural link region of the steerable catheter tube 31, which produces a predefined or predefinable deflection of the flexural link region.

However it is also possible, of course, to choose other embodiments as the operating means 33 of the steering means 30 for deflecting the steerable catheter tube 31 or a flexural link region of the steerable catheter tube 31.

In the exemplary embodiment of the steering means 30 illustrated in FIG. 3b, the proximal end section of the steerable catheter tube 31 of the steering means 30 terminates in a port section 36 of the steering means 30. The port section 36 of the steering means 30 serves for introducing the catheter tip 11 and catheter shaft 12 of the delivery means 10 into the steerable catheter tube 31. For this purpose, the port section 36 of the steering means 30 has a lumen defining a passageway which extends through the port section 36, the distal end section of the passageway being aligned with the proximal end section of the steerable catheter tube 31.

As illustrated in FIG. 1, the catheter tip 11 and catheter shaft 12 of the delivery means 10 is introducible (via the port section 36 of the steering means 30) into the steerable catheter tube 31. The catheter shaft 12 of the delivery means 10 and particularly the first catheter tube of the catheter shaft of the delivery means 10 is moveable relative to the steerable catheter tube 31. In particular, the steerable catheter tube 31 terminates proximal to the catheter tip 11 wherein the cross-section of proximal end section of the introducer sheath shall be substantially the same as or slightly larger than the cross-section of the flared section provided at the proximal end of the first catheter tube.

As illustrated in FIG. 1, the exemplary embodiment of the delivery system 100 is also provided with an introducer 40 having an introducer sheath 41. The introducer sheath 41 has a cross-section greater than the cross-section of the steerable catheter tube 31 of the steering means 30.

The introducer sheath 41 serves as guiding means when introducing the delivery means 10 and/or the steering means 30 and, in particular, the steerable catheter tube 31 of the steering means 30 into the patient's vasculature. In more detail, the introducer sheath 41 defines a passageway through which the catheter tip 11 and catheter shaft 12 of the delivery means 10 and/or the steerable catheter tube 31 of the steering means 30 may be guided to the implantation side within the patient's body.

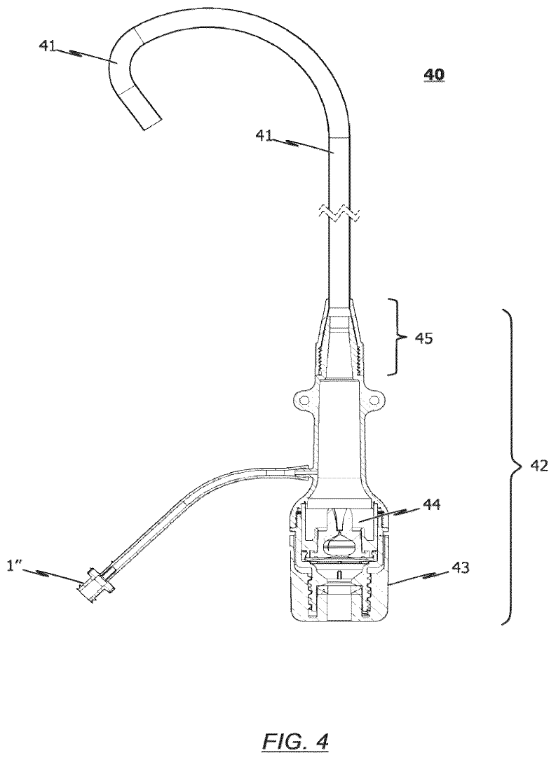

A sectional-side view of one embodiment of the introducer 40 is illustrated in FIG. 4.

Hence, the introducer sheath 41 has a distal end, a proximal end and a passageway extending there between. The introducer sheath 41 has a length such that the distal end of the introducer sheath 41 terminates proximal to the catheter tip 11 of the delivery system, when the catheter shaft 12 and the catheter tip 11 of the delivery system 100 has been fully introduced into the introducer sheath 41.

When at least a section of the catheter shaft 12 of the delivery means 10 or at least a section of the steerable catheter tube 31 of the steering means 30 has been introduced into the passageway defined by the introducer sheath 41, the introducer sheath 41 is disposed concentrically and coaxially with the section of the catheter shaft 12 of the delivery means 10 or the steerable catheter tube 31 of the steering means 30.

In any case, however, the catheter shaft 12 of the delivery means 10 and/or the steerable catheter tube 31 of the steering means 30 is moveable relative to the introducer sheath 41. In particular, the introducer sheath 41 terminates proximal to the catheter tip 11 wherein the cross-section of proximal end section of the introducer sheath 41 shall be substantially the same as or slightly larger than the cross-section of the steerable catheter tube 31.

The proximal end section of the introducer sheath 41 is connected to an introducer port 42. The introducer port 42 serves for providing access to the introducer sheath 41 of the introducer 40 required for delivering the delivery means 10 or the steerable catheter tube 31 of the steering means 30 into the introducer sheath 41.

The introducer port 42 may comprise a base member 43 which is configured to be manually fixable to the steerable catheter tube 31 is introduced into the passageway defined by the introducer sheath 41, or to be manually fixable to the handle 32 of the steering means 30, when the steerable catheter tube 31 of the steering means 30 is introduced into the passageway defined by the introducer sheath 41.

As can be seen from FIG. 4 the introducer port 42 is provided with a sealing arrangement 44 for preventing leakage of fluid, in particular blood, from the introducer 40, when the introducer sheath 41 is introduced into the patient's vasculature.

Moreover, in the exemplary embodiment of the introducer 40 illustrated in FIG. 4, the proximal end section of the introducer sheath 41 terminates in a crimping section 45 distal to the sealing arrangement 44 of the introducer port 42. The crimping section 45 of the introducer 40 serves for crimping at least a middle section of a heart valve fixed to the catheter tip 11 of the delivery means 10 during the introduction of the catheter tip 11 into the introducer sheath 41. As described with respect to FIG. 2c only the distal and proximal end sections of the heart valve are fixed to the catheter tip 11 of the delivery means 10 by means of the first and second retaining means 15, 16, wherein no dedicated retaining means is allocated to the middle section of the heart valve between the distal and proximal end sections of the heart valve.

For crimping the middle section of the heart valve during the introduction of the catheter tip 11 into the introducer sheath 41, the catheter tip 11 passes through the crimping section 45 of the introducer 40 thereby further reducing the diameter of the middle section of the heart valve fixed to the catheter tip 11. As illustrated in FIG. 4, the crimping section 45 may comprise a conical tubular member having an inner diameter which decreases in the distal direction of the tubular member.

The introducer sheath 41 may be of a thin material such as to allow bending of the introducer sheath 41 in order to follow the tortuous pathway from the femoral artery to the implant site. The introducer sheath 41 material, however, shall have sufficient stiffness in order to mechanically avoid kinking of the flexible sections of the distal portion of the catheter shaft 12 during insertion of the catheter tip 11.

An inlet 1, 1′, 1″ may be provided at a proximal end sections of the steering means 30, the introducer 40 and/or the delivery means 10 for injection of fluids, if necessary. Furthermore, a check valve may be provided at the proximal end section of the introducer sheath 41 to prevent fluid from leaking out of the introducer sheath 41.

The introducer sheath 41 may have a length sufficient to protect the inner wall of the blood vessel through which the catheter tip 11 passes. In addition, a separate introducer system (not belonging to the catheter system) may be provided. The introducer system then may serve as a portal for passing the complete catheter system from the catheter tip 11 to the catheter shaft 12 into the patient's body and up to the heart.

In addition, the introducer sheath 41 reduces the compression force exerted on the first catheter tube that is inserted through the introducer sheath 41. This increases manoeuvrability of the steerable catheter tube 31 and the catheter shaft 12 of the delivery means 10 throughout the procedure. A consequence thereof is that any frictional force is reduced. Moreover, moving the catheter tip 11 after it has been advanced through the vascular system of a patient, is greatly improved while at the same time lowering the risk of injury of the patient.

As will be appreciated, the introducer sheath 41 will be of a size, i.e. has an outer diameter, which will permit insertion in a patient's blood vessel (artery or vein) which is used for moving the stent transarterially or via a vein to an insufficient heart valve.

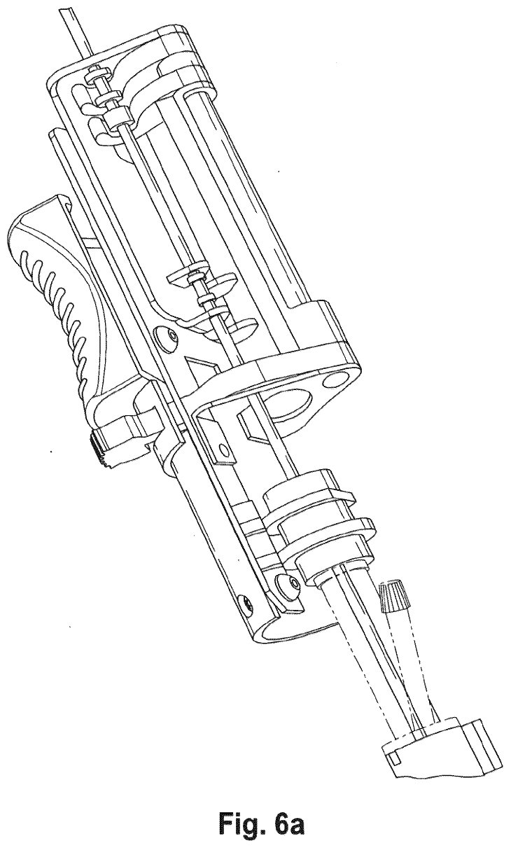

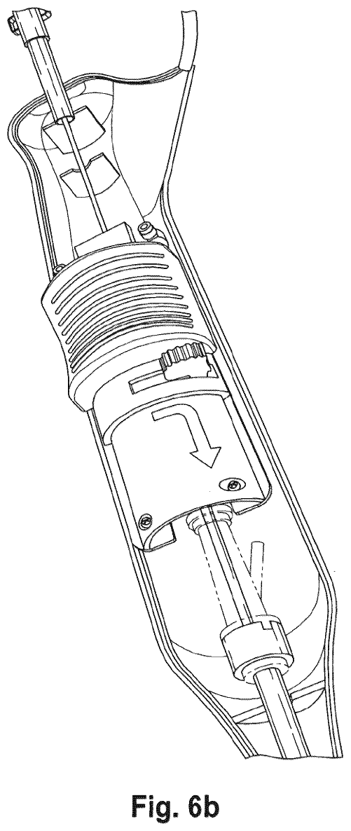

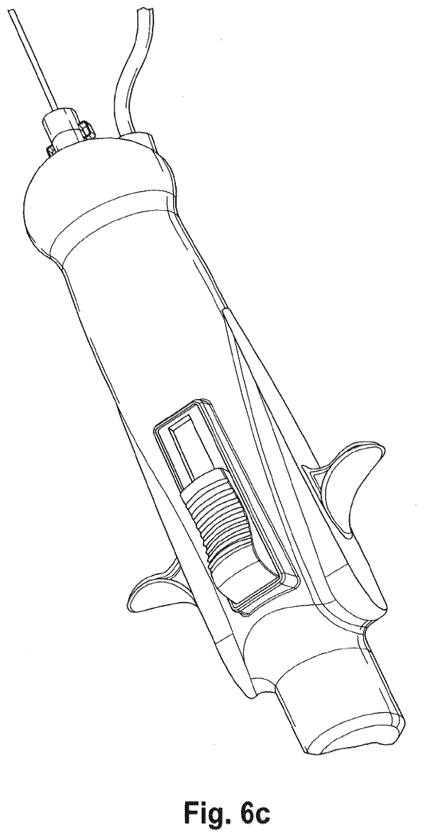

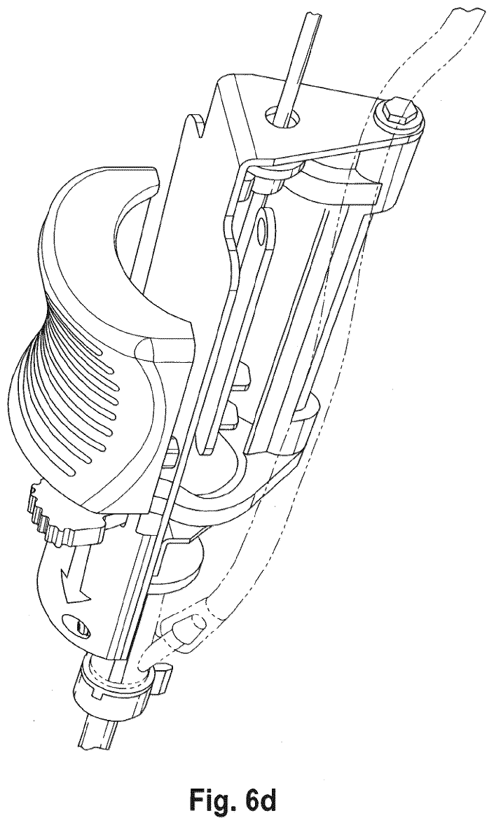

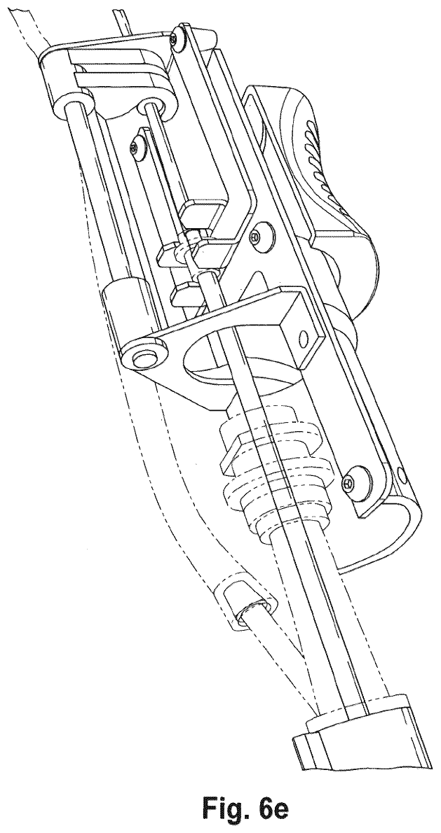

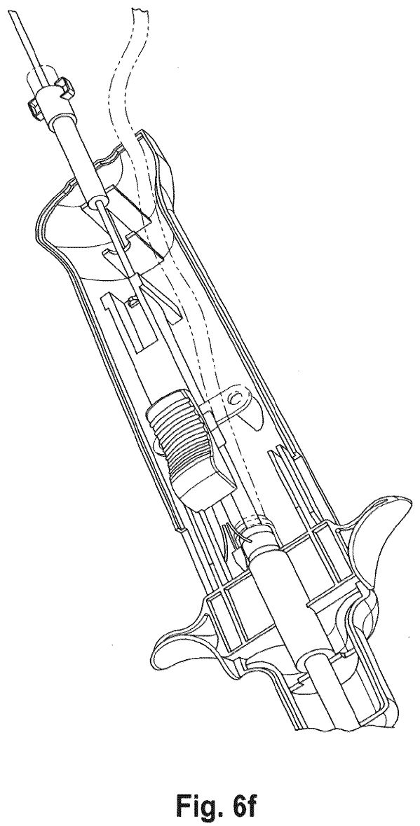

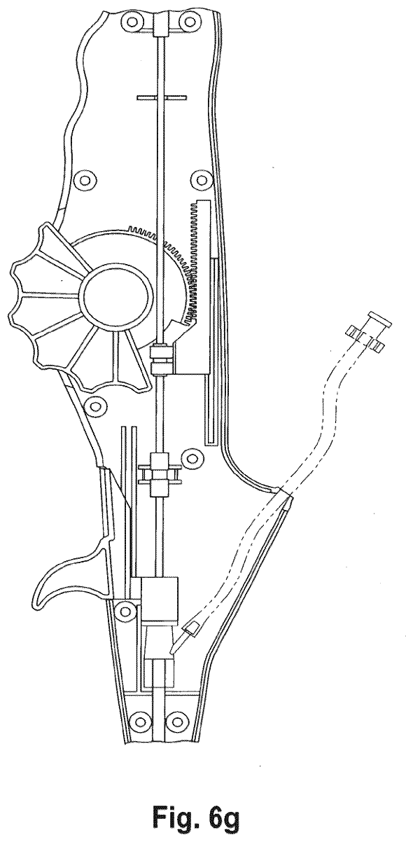

The FIGS. 6a to 6i represent sectioned side elevations of components of the delivery means of FIG. 2a.

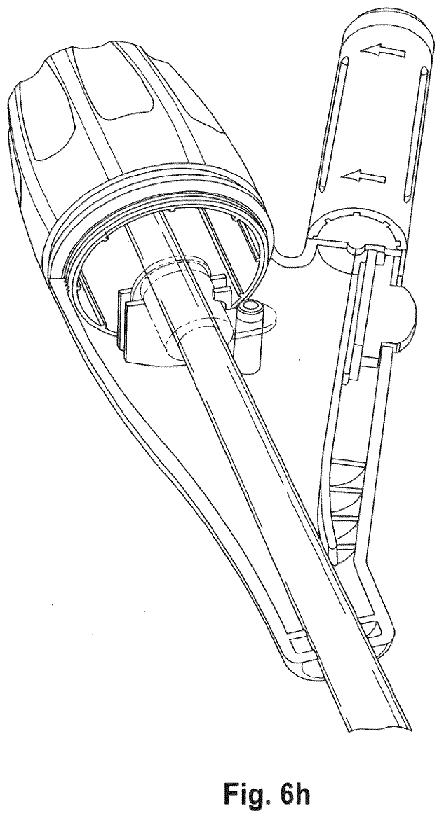

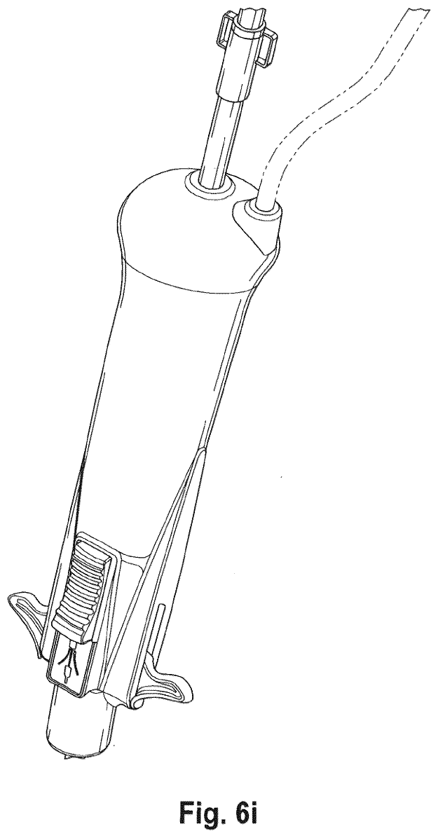

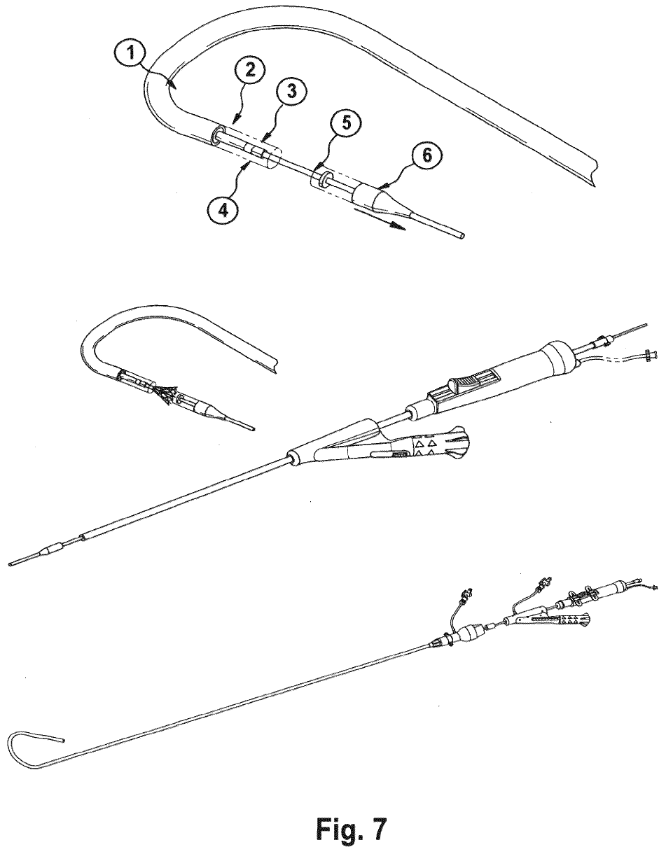

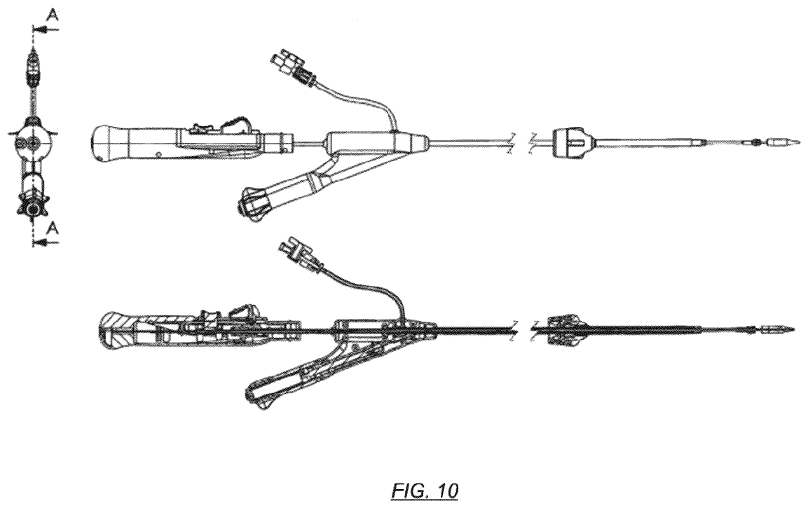

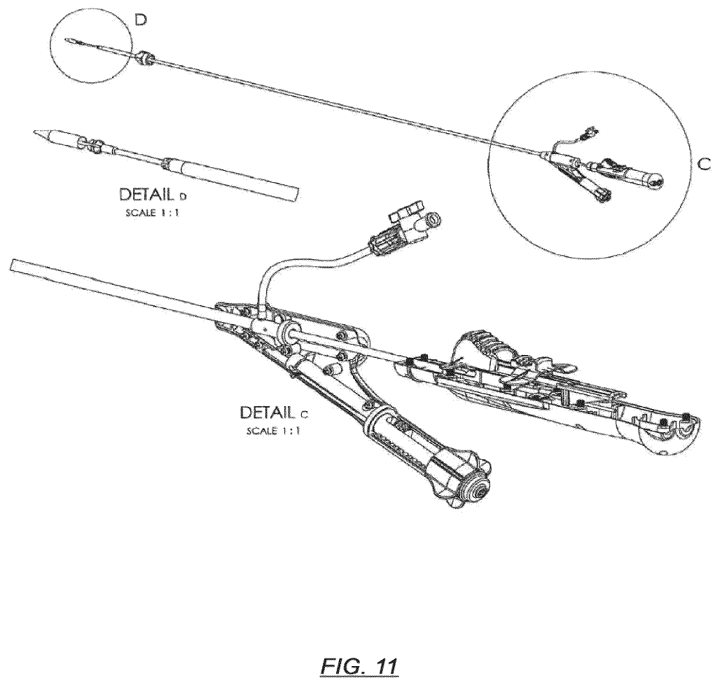

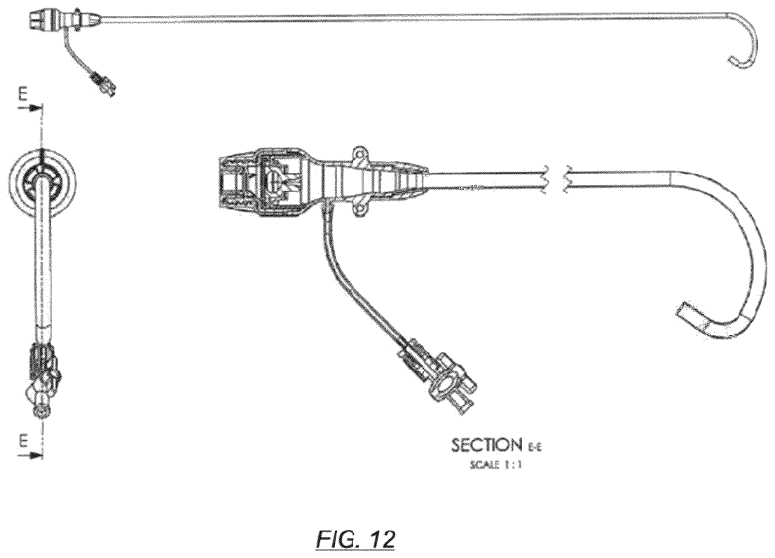

The FIGS. 7 to 12 represent further embodiments of the present disclosure.

In one aspect, embodiments of the present disclosure relate to a catheter system, comprising:
i) a steering means; and
ii) a delivery means for a heart valve, optionally balloon expandable or self-expandable, wherein the steering means and the delivery means are coaxially and rotationally independently movable.

In an embodiment, said catheter system is compatible with an introducer for intravascular, optionally transfemora (TF), applications or can be modified to be suitable for transapical (TA) delivery of a replacement heart valve. In such a TA system and means the direction in which the heart prosthesis is releaseably connected with the delivery means in turned by 180° compared to the TF application.

In some embodiments, the catheter system according to the present disclosure is made such that the introducer comprises an introducer sheath which extends or is extendable through the vascular duct and optionally proximal to the target site of the heart and optionally wherein the introducer sheath exhibits a crimping section.

The catheter system according to embodiments of the present disclosure may have a first and a second retaining means at the distal end of the catheter system to reversibly secure the heart valve to the delivery means.

Optionally the catheter system according to embodiments of the present disclosure is characterized in that the first retaining means reversibly secures the distal end of the heart valve to the delivery means and the second retaining means reversibly secures the proximal end of the heart valve to the delivery means.

In an embodiment, the mid-section of the heart valve is not covered by a sleeve means.

It thus rests flexible in terms of diameter and the tissue is not pressured by way of a crimping procedure that results in tension onto the pericard valve material. In this manner the heart valve is only crimped during the deployment procedure and thus the time during which the replacement heart valve is crimped to its smallest diameter is kept relatively short.

Accordingly, in at least one embodiment of a catheter system according to the present disclosure the diameter of the secured heart valve varies over its length, and preferably the heart valve is compressible over its length to the inner diameter of the introducer sheath.

In at least one embodiment thus the catheter system according to the present disclosure the inner diameter if the introducer sheath can be chosen as required by the respective application and may be less than 24 French, for example, less than 20 French, less than 18 French, and even less than 16 French.

In at least one embodiment the introducer sheath has a pre-shaped, preferably a curved, configuration.

The skilled person will appreciate that materials usually applied in catheter and delivery systems can also be used in embodiments according to the present disclosure. For example, in a catheter system according to an embodiment of the present disclosure the introducer sheath is characterized by a flexible polymer, a hydrophilic coating, a PTFE liner, coil reinforcement and/or braid reinforcement.

The replacement heart valve may be attached with useful meant to the delivery means which are compatible with the other parts of the device. In an embodiment the heart valve is attached to the delivery means by way of holding means wherein the heart valve and the delivery means exhibit complementary shapes. For example, the heart valve may have male connector elements at the distal and proximal end to engage with female connector elements on the delivery means.

In at least one embodiment according to the present disclosure in the catheter system the delivery means and steering means are operated by one or two means, optionally one or two handles. The steering means may exhibit means to deflect the shaft of the delivery catheter from a relaxed configuration into a curved configuration.

In at least one embodiment of the catheter system according to the present disclosure the delivery means and steering means are releasably connectable.

In another aspect, embodiments of the present disclosure relate to a delivery system for a heart valve comprising i. a steering means; ii. a delivery means for a heart valve and iii. an introducer sheath wherein the steering means, the delivery means and the introducer sheath are coaxially and circumferentially independently movable.

In an exemplary embodiment the introducer sheath extends or is extendable through the vascular duct and optionally proximal to the heart.

The different parts and sections of the delivery system may be adapted to each other and made in a way that they are compatible in their constructive and functional characteristics. In at least one embodiment, the steering means, the delivery means and the introducer sheath are at least sectionally, partially or substantially completely arranged coaxially to each other. The steering means, the delivery means and the introducer sheath may be coaxially and rotationally independently movable.

In the delivery system according to some embodiments of the present disclosure, the delivery means and steering means can be simultaneously or independently introducible into the introducer sheath.

In the delivery system according to embodiments of the present disclosure the introducer sheath can exhibit a crimping section for reducing the diameter of the heart valve attached to the delivery means. Such a crimping section has the advantage that the replacement heart valve is only crimped over its entire length to its minimal diameter during the delivery and deployment procedure which leads to less stress impact onto the pericard tissue of the replacement heart valve.

In another aspect, embodiments of the present disclosure relate to a method for the delivery of a heart valve to the heart of a patient comprising the steps i. in a first step placing an introducer comprising an introducer sheath into a patient's vasculature; ii. in a second step introducing a catheter system having fixed thereto a self-expanding or balloon expandable heart valve through the introducer into the patient's vasculature; iii. in a third step positioning the heart valve proximal to the target site in the patient's heart, optionally positioning the heart valve substantially central in the target area; iv. in a fourth step positioning the heart valve at the target site; v. in a fifth step partially releasing the heart valve from the catheter system; vi. in a sixth step fully releasing the heart valve from the catheter system.

In some methods according to embodiments of the present disclosure at least one segment of the self-expanding heart valve is compressed from a first diameter to a second diameter that is smaller than the first diameter when the self-expanding heart valve is passed through the introducer sheath, preferably the crimping section.

In another aspect of embodiments of the present disclosure, at least one segment of the self-expanding heart valve expands from a first diameter to a second diameter that is larger than the first diameter when the self-expanding heart valve exits the distal end of the introducer sheath.

In another aspect, embodiments of the present disclosure relate to a method of loading a self-expanding or balloon expandable heart valve onto a delivery means of a catheter system having at least one shaft and at least one outer sheath and/or is characterized as described above and throughout the specification and comprising or consisting of:
compressing the heart valve radially and placing the compressed heart valve into a tubular retainer, the retainer preventing the self-expanding valve from expanding radially,
passing the inner shaft of the delivery system through the central opening of the compressed heart valve,
attaching the distal tip to the shaft of the delivery system,
advancing the outer sheath onto the compressed heart valve to prevent radial expansion of at least part of the heart valve, and
removing the tubular retainer from the compressed self-expanding heart valve.

In the following further alternative and/or exemplary embodiments will be described.

In at least one embodiment the present disclosure relates to a delivery system for a prosthesis, e.g. a heart valve, which consists of or comprises in general three components. The three components each consist of a handle wherein each handle is characterized by additional parts as follows.

A port handle comprises a pre-shaped tubular component which can be introduced through the vasculature, e.g. transfemoral, of a patient starting femoral up to proximal to the heart. This introducer sheath has a length of 50 to 150 cm, such as 100 to 120 cm. The tubular element may have a pre-shaped bent or may be designed in a manner so that it easily bents around the main artery proximal the heart. A balloon device can be introduced through this handle/introducer sheath which represents a first step in the prosthesis delivery procedure. This handle exhibits a twist grip and is positioned at the upper leg and is functional to introduce the other components of the delivery system. The tubular component can be pushed through the port handle or is fixed thereto. The tubular component is made from a compatible polymer material reinforced with wire and may include PTFE or Teflon coated at the inside. The port handle may exhibit a valve or other means for closing the hollow entry tube which serves to introduce the introducer sheath and the catheter component of the system. The inner diameter of the introducer sheath is less than 25 French, such as less than 20 French, less than 18 French or has a diameter of 20, 18 or 16 French.

The introducer sheath can be coupled to the second handle (introducer sheath handle) by way of which one can push the introducer sheath combined with the introducer sheath handle. The introducer sheath handle exhibits a wire of similar means in a manner so that the introducer sheath is steerable. In this manner during the deployment of the prosthesis the introducer sheath can be bent or directed in a defined position which will facilitate the deployment procedure. The first and second handles can exhibit a means to releasable connect them to each other.

A third handle represents the catheter component of the delivery system. It comprises a handle, a catheter shaft, a distal capsule for releasable connecting the prosthesis, a tubing for flushing including a closing means. The handle exhibits control means for connecting the prosthesis and releasing it.

Any component or all components can be introduced by way of a guide wire.

The delivery procedure can follow the steps of opening the femoral vasculature as access and introducing a port and/or a guide wire. The port handle and the introducer sheath is positioned at the upper leg and introduced ascending the aorta. In a next step a balloon device is introduced through the port and the introducer sheath for widening by way of balloon expansion the target site. After this task is performed the balloon device is retrieved. In a next step the catheter is introduced through the introducer sheath. The prosthesis is preloaded onto the catheter at the distal capsule. The prosthesis is connected with its ends at defined mounting positions which represent male/female connections which allow the controlled release of the prosthesis at the target site in a step-wise manner. Accordingly, the prosthesis is connected with the catheter so that the connections represent a small diameter and the prosthesis is otherwise not connected and also not covered by way of a sheath or other covering. Thus the diameter of this middle part of the prosthesis is due to the outward tension of the stent material larger than at the connection sites. An advantage of this design is that a small diameter of the loaded prosthesis can be achieved. In the course of introducing the prosthesis loaded onto the catheter capsule into and through the introducer sheath the prosthesis will be crimped to its final diameter pre-defined by the inner diameter of the introducer sheath. The material design of the introducer sheath facilitates the crimping and also the progression through this sheath.

The prosthesis can now be approached in direction to the target site using the introducer sheath and catheter. Another advantage of the delivery system is that the catheter and the introducer sheath are both coaxially and rotationally independently movable. Moreover, by way of the steering means of the second handle the introducer sheath can be bended and thus facilitate to maneuver the prosthesis through the vasculature to its final target site. Another advantage of a system according to embodiments of the present disclosure is that the catheter handle and the second handle of the introducer sheath with the steering means can be moved and operated independently.

The release procedure can be managed by a reversible two-step process. The release process is initiated once the feelers of the prosthesis are placed inside the cusps of the native aortic valve. In a first step the proximal connecting means is pushed in a forward direction/proximal direction. This will release the proximal end of the prosthesis. The catheter of the prosthesis may exhibit visualization means for better visual control. In a second step, the distal connecting means may be retracted or pulled back by way of a control means on the catheter handle to release the distal end of the prosthesis and disconnect the entire prosthesis from the catheter. The control means can be designed as independent means for each connecting means or in a connected manner.

Thus the advantages of embodiments of the present disclosure are that the loading and in particular crimping of the prosthesis is achieved by way of the introducer sheath and thus the crimping procedure is combined with the introduction of the prosthesis into the vasculature which facilitates the process. This implies the advantage that a separate cover means to crimp the prosthesis beforehand is not necessary with the effect and additional advantage that the tissue of the prosthesis is more smoothly and less vigorously crimped. This implies a lower risk of damages to the tissue. Moreover, the crimping procedure is facilitated and the middle part of the prosthesis which is usually the thickest part of a valve prosthesis needs not be crimped by a special device or by hand. Another advantage is the steering mechanism independent from the catheter handle. The extension of the port by way of the introducer sheath for the delivery of heart prosthesis is novel and has the advantages as above described.

A further advantage is that the bending can be managed independently from the delivery capsule. Thus all parts like port, handles, capsule, steerable means and prosthesis are all independently movable and coaxially and rotationally movable which facilitates the deployment of the prosthesis at the target site. Moreover, the prosthesis may be deployed by a two-step procedure and is repositionable. The procedure may also be aborted during the delivery procedure.

The combination of the different components of the delivery system may help to achieve certain advantages and superior delivery and deployment results in a simple and safe manner.

The release of the heart valve prosthesis from the delivery means can occur in a 2-step deployment way.

The skilled person will appreciate that the catheter system, the delivery system, the method for loading and the method for the delivery of the heart valve to the heart may be combined with suitable devices and methods or steps to prepare the target site in the heart for the deployment of the replacement heart valve prosthesis. Accordingly, a balloon device or balloon system can be applied before the delivery of the replacement heart valve to expand the target site within the heart. This system may be introduced by way of the port and/or introducer sheath according to embodiments of the present disclosure which exhibits the advantage that the port and introducer sheath can be positioned in the patient's vasculator entrance femorally and only one access is necessary to perform the dilatation of the target site by way of balloon expansion. After the balloon device is retrieved the replacement prosthesis can be applied as described above.

Parts and devices that are introduced can be guided by a guide wire known in the art and made from materials as usually applied in the field.

It can be advantageous if the tip of the catheter device is made of a soft or semi firm material and/or which is bendable in order to easily pass through the vasculator of the patient. Known materials can be used for such a flexible tip.

It is emphasized that the combination of the introducer sheath with the delivery catheter part according to embodiments of the present disclosure does not only simplify the loading and delivery procedure but an advantage is also that less layers of heart valve prosthesis coverage is needed and thus the diameter and crimping diameter can be reduced or/and the forces applied to the prosthesis is influenced in an advantageous manner. Thus less impact as compared in conventional devices and loading procedures can be expected which implies the advantage of les damage and a longer lifespan of the replacement heart valve.

In view of the particular construction characteristics of various catheters and delivery devices according to embodiments of the present disclosure one can denote two steps to the delivery procedure. The first step being the placement and the port, introducer sheath and loading of the replacement heart valve and the second step the deployment of the heart valve to the target site. The deployment as such is in turn a two-step sequence of steps with a first release step of part of the replacement heart valve and in a second step the final and complete release and final positioning of the replacement heart valve at the target site. In between these steps a retrieval of the replacement heart valve is still possible and offers the option of repositioning and complete retrieval and stop of the deployment procedure.

In accordance with an aspect, the present disclosure provides a catheter system for introducing an expandable heart valve into the body of a patient, the catheter system comprising delivering means for the heart valve.

In some aspects of the present disclosure, the delivering means comprises a catheter tip and a catheter shaft. The catheter tip of the delivery means has a seat portion for accommodating the heart valve to be introduced into the patient's body in its collapsed or crimped state. The catheter tip has further holding means for realisably fixing the heart valve to the catheter tip.

The seat portion of the catheter tip is constituted by a first retaining means and a second retaining means. In some embodiments of the present disclosure, the first retaining means and second retaining means may be constituted by a first sleeve-shaped member and a second sleeve-shaped member.

The first retaining means of the catheter tip serves for reversibly securing a distal end section of the heart valve to the delivery means and, in particular, to the catheter tip of the delivery means. On the other hand, the second retaining means of the catheter tip serves for reversibly securing a proximal end section of the heart valve to the delivery means and, in particular, to the catheter tip of the delivery means.

The first and second retaining means are moveable relative to each other and also relative to the holding means of the catheter tip.

According to some embodiments of the present disclosure, the catheter shaft of the delivery means comprises first force transmitting means and second force transmitting means.

A distal end section of the first force transmitting means is connected or connectable to the first retaining means of the catheter tip and a proximal end section of the first force transmitting means is connected or connectable to a first operating means of a handle of the delivery means. A distal end section of the second force transmitting means is connected or connectable to the second retaining means of the catheter tip and a proximal end section of the second force transmitting means is connected or connectable to a second operating means of the handle of the delivery means.

According to some embodiments of the present disclosure, the handle of the delivery means has at least one first operating means and at least one second operating means with which the catheter tip of the delivery means may be appropriately manipulated so that an expandable heart valve secured to the catheter tip may be released from the catheter tip in steps or in a defined or definable sequence of events.

In accordance with some embodiments of the present disclosure, the catheter tip has first and second retaining means—for example in the form of sleeve-shaped members which may be manipulated with the handle of the delivery means. These retaining means are used for releasably and reversibly securing a distal and proximal end section of the heart valve to the catheter tip.

In some embodiments of the present disclosure, the first retaining means serves for releasably and reversibly securing first functional components of the heart valve, for example retaining hoops of a stent or alternatively positioning hoops of a stent, while the second retaining means serves for releasably and reversibly securing second functional components of the heart valve, for example, positioning hoops of a stent or alternatively for accommodating retaining hoops of a stent.

In relation to the handle of the delivery means, in some embodiments it is preferably provided that, on one hand, the first operating means cooperate with the first retaining means of the catheter tip so that, on actuation of the first operating means, a previously definable longitudinal displacement of the first retaining means may be effected relative to the holding means. On the other hand, the second operating means cooperates with the second retaining means of the catheter tip so that a previously definable longitudinal displacement of the second retaining means may be affected relative to the holding means.

In accordance with some embodiments of the present disclosure, the first and second retaining means only serve for securing the distal and proximal end sections of the heart valve to the catheter tip. In case the first and second retaining means are configured as first and second sleeve-shaped members, these sleeve-shaped members have a length such that a gap is between the first and second sleeve-shaped members when securing the distal and proximal end sections of the heart valve to the catheter tip.

In accordance with some embodiments of the present disclosure, the first force transmitting means of the delivery means is constituted by a first catheter tube defining a first lumen, and the second force transmitting means of the delivery means is constituted by a second catheter tube defining a second lumen. The second catheter tube has a cross-section less than the cross-section of the first catheter tube. The first catheter tube is disposed concentrically and coaxially with the second catheter tube and the second catheter tube is received within the first lumen defined by the first catheter tube.

Contrary to the first and second sleeve-shaped members of the catheter tip, however, the holding means of the catheter tip is not moveable relative to the handle of the delivery means. Rather, the holding means is connected to the handle of the delivery means by using a holding tube having a distal end connected to the holding means and a proximal end connected to a body of the handle of the delivery means. The holding tube has a cross-section less than the cross-section of the first catheter tube. In particular, the first catheter tube is disposed concentrically and coaxially with both, the second catheter tube on the one hand and the holding tube on the other hand.

In some embodiments, the holding tube has a cross-section less than the cross-section of the first catheter tube and greater than the cross-section of the second catheter tube such that the holding tube is received within the first lumen defined by the first catheter tube and the second catheter tube is received within a passageway defined by the holding tube. The passageway defined by the holding tube has a diameter sufficient to accommodate the second catheter tube such that the second catheter tube is moveable relative to the holding tube.

The second lumen defined by the second catheter tube has a diameter sufficient to accommodate a guide wire. The second catheter tube is made from a rigid material including, for example, nitinol, stainless steel or a rigid plastic material. The material of the distal end section of the second catheter tube may have an increased flexibility compared to the material of the proximal end section in order to allow the distal end section of the catheter shaft to pass the aortic arch during insertion of the catheter tip.

In some embodiments of the present disclosure, the distal end section of the second catheter tube terminates in a soft catheter end tip having an atraumatic shape. The soft catheter end tip is provided with a channel aligned with the second lumen defined by the second catheter tube such that a guide wire accommodated within the second lumen of the second catheter tube may pass through the channel of the soft catheter end tip. The second sleeve-shaped member of the catheter tip is connected to the soft catheter end tip such that the opened end of the second sleeve-shaped member faces in the proximal direction opposite to the direction of the soft catheter end tip and to the second catheter tube.

In some embodiments, the holding tube is made of a rigid material, for example, a rigid plastic material, stainless steel or Nitinol. The distal end of the holding tube terminates in the holding means which is also made of a rigid material, for example, a rigid plastic material or stainless steel. The passageway defined by the holding tube is aligned with a channel which passes through the holding means. In this way, the second catheter tube is accommodated in the passageway of the holding tube and the channel of the holding means such as to be moveable relative to the holding tube and the holding means.

The holding tube is provided for connecting the holding means to the handle of the delivery means. For this purpose, the holding tube has a distal end connected to the holding means and a proximal end connected to a body of the handle of the delivery means.

In some embodiments, the first catheter tube is preferably made of a bendable but axially rigid material. For example, the first catheter tube may be at least partly made of a braided or non-braided catheter tube. Hence, the first catheter tube may have a stiff braid reinforced body similar to the catheter body described in U.S. Pat. No. 4,665,604 which is incorporated herein by reference.

The first catheter tube shall be adapted to transfer compression and tension forces from the first operating means of the handle of the delivery means to the first retaining means of the catheter tip without overly changing of its total length. The distal end of the first catheter tube terminates at a flared section as the transition to the section defining the first retaining means of the catheter tip.

The flared section and the first retaining means may be formed integrally and may be connected to the distal end section of the first catheter tube.

Alternatively, the first retaining means and the flared section of the first catheter tube may be all of the same material and originating from the same raw tube prior to a widening process so that the flared section and the first retaining means are the same elements.

In accordance with another aspect of the present disclosure, the catheter system further comprises steering means. The steering means for guiding the delivery means and, in particular, the catheter tip of the delivery means when advancing the catheter tip through the patient's vasculature.

In some embodiments of the present disclosure, the steering means comprises a steerable catheter tube having a proximal end section and a distal end section. The proximal end section of the steerable catheter tube is connected or connectable with a handle of the steering means.

The handle of the steering means is provided with operating means, for example in the form of a rotatable knob or wheel, by means of which a flexural link region of the steerable catheter tube can be controlled.

In accordance with other embodiments disclosed herein, the material of the distal end section of the steerable catheter tube may have an increased flexibility compared to the material of the proximal end section. In this way, the distal end section of the steerable catheter tube may easily pass, for example, the aortic arch during insertion of the steerable catheter tube.

In some embodiments disclosed herein, the handle of the steering means is provided with operating means by means of which a flexural link region of the steerable catheter tube can be controlled, wherein the operating means preferably has a detent device to allow a set deflection of the flexural link region of the steerable catheter tube to be fixed. For example, it is possible to provide a suitable catch mechanism on the operating means, which cooperates with a body of the handle of the steering means. In particular, it is possible for the flexural link region of the steerable catheter tube to be connected to the operating means of the steering means by way of a control wire whereby, on an actuation of the operating means via the control wire a tensile forces is exerted on the flexural link region of the steerable catheter tube, which produces a predefined or predefinable deflection of the flexural link region.

However it is also possible, of course, to choose other embodiments as the operating means of the steering means for deflecting the steerable catheter tube or a flexural link region of the steerable catheter tube, in case the steerable catheter tube is provided with such a flexural link region.

In accordance with some embodiments disclosed herein, the proximal end section of the steerable catheter tube of the steering means terminates in a port section of the steering means or is connected with a port section of the steering means. The port section of the steering means serves for introducing the catheter tip and catheter shaft of the delivery means into the steerable catheter tube. For this purpose, the port section of the steering means has a lumen defining a passageway which extends through the port section, the distal end section of the passageway being aligned with the proximal end section of the steerable catheter tube.

In some embodiments, the port section of the steering means is preferably integrated in or connected with the handle of the steering means.

The catheter tip and catheter shaft of the delivery means is introducible (via the port section of the steering means) into the steerable catheter tube. The catheter shaft of the delivery means and particularly the first catheter tube of the catheter shaft of the delivery means is moveable relative to the steerable catheter tube. In particular, the steerable catheter tube terminates proximal to the catheter tip wherein the cross-section of proximal end section of the introducer sheath shall be substantially the same as or slightly larger than the cross-section of the flared section provided at the proximal end of the first catheter tube.

The proximal end section of the steering means and, in particular, the proximal end section of the port section of the steering means is releasably connectable to the handle of the delivery means.

According to an aspect of the present disclosure, the catheter system further comprises an introducer having an introducer sheath. The introducer sheath has a cross-section greater than the cross-section of the steerable catheter tube of the steering means.

The introducer sheath serves as guiding means when introducing the delivery means and/or the steering means and, in particular, the steerable catheter tube of the steering means into the patient's vasculature. In more detail, the introducer sheath defines a passageway through which the catheter tip and catheter shaft of the delivery means and/or the steerable catheter tube of the steering means may be guided to the implantation side within the patient's body.

The introducer sheath has a distal end, a proximal end and a passageway extending there between. The introducer sheath has a length such that the distal end of the introducer sheath terminates proximal to the catheter tip of the delivery system, when the catheter shaft and the catheter tip of the delivery system has been fully introduced into the introducer sheath.

When at least a section of the catheter shaft of the delivery means or at least a section of the steerable catheter tube of the steering means has been introduced into the passageway defined by the introducer sheath, the introducer sheath is disposed concentrically and coaxially with the section of the catheter shaft of the delivery means or the steerable catheter tube of the steering means.

In any case, however, the catheter shaft of the delivery means and/or the steerable catheter tube of the steering means is moveable relative to the introducer sheath. In particular, the introducer sheath terminates proximal to the catheter tip wherein the cross-section of proximal end section of the introducer sheath shall be substantially the same as or slightly larger than the cross-section of the steerable catheter tube.

The proximal end section of the introducer sheath is connected to an introducer port. The introducer port serves for providing access to the introducer sheath of the introducer required for delivering the delivery means or the steerable catheter tube of the steering means into the introducer sheath.

The introducer port may comprise a base member which is configured to be manually fixable to the handle of the delivery means, when the catheter shaft of the delivery means is introduced into the passageway defined by the introducer sheath, or to be manually fixable to the handle of the steering means, when the steerable catheter tube of the steering means is introduced into the passageway defined by the introducer sheath.

In accordance with some embodiments disclosed herein, the introducer port is provided with a sealing arrangement for preventing leakage of fluid, in particular blood, from the introducer, when the introducer sheath is introduced into the patient's vasculature.

In some embodiments disclosed herein, the proximal end section of the introducer sheath terminates in a crimping section distal to the sealing arrangement of the introducer port. The crimping section of the introducer serves for crimping at least a middle section of a heart valve fixed to the catheter tip of the delivery means during the introduction of the catheter tip into the introducer sheath. As already mentioned above, according to embodiments disclosed herein, preferably only the distal and proximal end sections of the heart valve are fixed to the catheter tip of the delivery means by means of the first and second retaining means, wherein no dedicated retaining means is allocated to the middle section of the heart valve between the distal and proximal end sections of the heart valve.

For crimping the middle section of the heart valve during the introduction of the catheter tip into the introducer sheath, the catheter tip passes through the crimping section of the introducer thereby further reducing the diameter of the middle section of the heart valve fixed to the catheter tip. In some embodiments disclosed herein, the crimping section may comprise a conical tubular member having an inner diameter which decreases in the distal direction of the tubular member.

The introducer sheath may be of a thin material such as to allow length deformation of the introducer sheath upon transfer of compression and tension forces. The introducer sheath material, however, shall have sufficient stiffness in order to mechanically avoid kinking of the flexible sections of the distal portion of the catheter shaft during insertion of the catheter tip.

In some embodiments of the present disclosure, the introducer sheath has a pre-shaped, preferably curved, configuration.

An inlet may be provided at a proximal end section of the steering means, the introducer and/or the delivery means for injection of fluids, if necessary. Furthermore, a check valve may be provided at the proximal end section of the introducer sheath to prevent fluid from leaking out of the introducer sheath.

The introducer sheath may have a length sufficient to protect the inner wall of the blood vessel through which the catheter tip passes. In addition, a separate introducer system (not belonging to the catheter system) may be provided. The introducer system then may serve as a portal for passing the complete catheter system from the catheter tip to the catheter shaft into the patient's body and up to the heart.

In addition, the introducer sheath reduces the compression force exerted on the first catheter tube that is inserted through the introducer sheath. This increases manoeuvrability of the steerable catheter tube and the catheter shaft of the delivery means throughout the procedure. A consequence thereof is that any frictional force is reduced. Moreover, moving the catheter tip after it has been advanced through the vascular system of a patient, is greatly improved while at the same time lowering the risk of injury of the patient.

The length of the introducer sheath depends on the length of the catheter shaft of the delivery means and will typically be between about 20 cm to 60 cm for transaortic (TA) access and 80 cm to 150 cm for transfemoral (TF) access. Those skilled in the art will appreciate, however, that all dimensions provided herein are intended as examples only, and that the introducer sheaths and catheter tubes of different dimensions may be substituted for a particular use.

As will be appreciated, the introducer sheath will be of a size, i.e. has an outer diameter, which will permit insertion in a patient's blood vessel (artery or vein) which is used for moving the stent transarterially or via a vein to an insufficient heart valve.

The introducer sheath may be capable of traversing tortuous pathways in the body of the patient without kinking. The introducer sheath may include an inner lubricious liner, an outer polymeric jacket, and a coil reinforcement between the inner and the outer layers. This introducer sheath may provide favourable flexibility without kinking or compression. One or more radiopaque bands or markers may be incorporated within the introducer sheaths material to allow precise location of the introducer sheaths distal end for positioning accuracy. Those skilled in the art will appreciate that other known materials may also be suitable for a particular purpose.

The catheter system is particularly adapted to deliver and implant a heart valve as described for example in the European Patent Application No. 07 110 318 or in the European Patent Application No. 08 151 963. In some embodiments of the present disclosure, a heart valve is accordingly used which comprises a stent and a heart valve prosthesis attached to the stent. The stent exhibits the following:
a first retaining region, to which the heart valve prosthesis can be attached;
an opposing, second retaining region with at least one retaining element, for example in the form of retaining eyes or in the form of retaining heads, whereby at least one retaining element of the stent can be put in releasable engagement with the holding means of the catheter tip of the delivery means;
at least one retaining hoop, to which the heart valve prosthesis can be fastened; and
at least one and, in some embodiments, preferably three positioning hoops, which are designed to engage in pockets of the native heart valve in the implanted state of the stent, thus to enable automatic positioning of the stent in the aorta of the patient.

In particular, a catheter system is disclosed herein, with which an expandable heart valve stent with a heart valve prosthesis attached to this stent can be advanced to the implantation site in a particularly simple way, for example via the aorta of a patient being treated (transarterially or transfemorally). In some embodiments, during transarterial or transfemoral access by the catheter system, the whole free cross-section available within the aorta is not completely filled up, since the catheter tip provided at the distal end region of the catheter system, in which the stent can be accommodated with the heart valve prosthesis, can be made sufficiently small with respect to its external diameter.

The expandable heart valve stent with the heart valve prosthesis attached to it can be fixed temporarily during implantation in a crimped state to the catheter tip of the delivery means.

The catheter system designed for transarterial or transfemoral access is therefore suitable for inserting a heart valve stent with a heart valve prosthesis attached to it, transarterially or transfemorally into the body of the patient; for example, the catheter tip of the delivery means of the catheter system is inserted via puncture of the A. femoris communis (inguinal artery).

In particular, with the catheter system designed for transarterial or transfemoral access, the catheter shaft of the delivery means may be designed so that it is both kink-resistant and flexible such that a bending radius of up to 4 cm, e.g., up to 3 cm, can be realised, at least at the distal end region of the catheter shaft.

Exemplary embodiments will be described with reference to the appended drawings below.

In the following section, additional alternative and/or exemplary aspects of the present disclosure will be illustrated:
1. An improved system for delivering a self-expanding implant to an implant site within a blood vessel of a patient consisting of an introducer sheath and a delivery catheter, the introducer sheath extending from outside of the body of the patient to the vicinity of the implant site, the delivery catheter having a first and a second retaining means at the distal end of the delivery catheter to reversibly secure the implant to the delivery catheter, the first retaining means reversibly secures the distal end of the self-expanding implant to the delivery catheter and the second retaining means reversibly secures the proximal end of the self-expanding implant to the delivery catheter, wherein the delivery catheter is passed through the introducer sheath to deliver the self-expanding implant to the implant site, and the central segment of the self-expanding implant is compressed from a first diameter to a second diameter that is smaller than the first diameter when the self-expanding implant is passed through the introducer sheath.
2. The system of aspect 1, wherein the implant is at least one of a stent, a stent graft, and a heart valve prosthesis.
3. The system of aspect 1, wherein the implant has male connector elements at the distal and proximal end to engage with female connector elements on the delivery catheter.
4. The system of aspect 3, where the male connector elements are eyelets and the female elements are recesses in the delivery catheter.
5. The system of aspect 1, wherein the introducer sheath has a hemostasis valve at the proximal end.
6. The system of aspect 1, wherein the retaining means consists of axially moveable sheaths that at least partially restrict radial expansion of the implant.
7. A process for delivering a self-expanding implant to an implant site within a blood vessel, comprising in combination: inserting an introducer sheath from outside the body through an opening in the body into a blood vessel; placing the distal end of the introducer sheath in the vicinity of the implant site; reversibly securing the distal end and the proximal end of the self-expanding implant to the distal end of a delivery catheter; inserting the delivery catheter with the self-expanding implant secured to the delivery catheter into the introducer sheath wherein the central segment of the self-expanding implant is compressed from a first diameter to a second diameter when inserted into the introducer sheath; advancing the delivery catheter through the introducer sheath until the self-expanding implant exits the distal end of the introducer sheath; and releasing the distal end and the proximal end of the self-expanding implant from the delivery catheter at the implant site.
8. A system for delivering an implant to an implant site within a blood vessel of a patient consisting of an introducer sheath, a delivery catheter, and a biasing member, wherein the introducer sheath extends from outside of the body of the patient to the vicinity of the implant site; the delivery catheter has retaining means to reversibly retain an implant at the distal end of the delivery catheter; the biasing member has means to deflect the shaft of the delivery catheter from a relaxed configuration into a curved configuration; wherein the delivery catheter is passed through the introducer sheath to deliver the implant to the implant site; and, the biasing member is axially and rotationally moveable inside the introducer sheath with respect to the introducer sheath and the delivery catheter.
9. The system of aspect 8, wherein the implant is a self-expanding or balloon expandable heart valve prosthesis.
10. The system of aspect 9, wherein the implant site is the native aortic or mitral valve.
11. The system of any one of the aspects herein, wherein the introducer sheath has a pre-shaped curved configuration.
12. A system for maneuvering an implant to an implant site within a blood vessel of a patient consisting of an introducer sheath, a delivery catheter, and a biasing member, which comprises, in combination: the delivery catheter having retaining means to reversibly retain an implant at the distal end of the delivery catheter; the biasing member having means to deflect the shaft of the delivery catheter from a relaxed configuration into a curved configuration; the delivery catheter is inserted into the introducer sheath and is axially and rotationally moveable within the introducer sheath; and the biasing member is inserted into the introducer sheath and is axially and rotationally moveable within the introducer sheath and is axially and rotationally moveable with respect to the delivery catheter.
13. A method of positioning an implant toward a target site within a blood vessel, comprising, the steps of: inserting an introducer sheath from outside the body through an opening in the body into a blood vessel; placing the distal end of the introducer sheath in the vicinity of the implant site; reversibly securing the implant to the distal end of a delivery catheter; placing a biasing member having means to actively deflect the shaft of the delivery system from a relaxed configuration into a curved configuration onto the shaft of the delivery system; inserting the delivery catheter with the implant and biasing member into the introducer sheath; advancing the delivery catheter through the introducer sheath until the implant exits the distal end of the introducer sheath; adjusting the axially and rotationally position of the biasing member within the introducer sheath; activating the biasing member to deflect the shaft of the delivery system into a desired bend configuration; adjusting the axially position of the delivery system to place the implant at the target site; and releasing the implant from the delivery system.
14. A method of loading a self-expanding heart valve onto a delivery catheter having an inner shaft, an attachable distal tip, and at least one outer sheath, comprising: compressing the self-expanding heart valve radially and placing the compressed self-expanding heart valve into a tubular retainer, the retainer preventing the self-expanding valve from expanding radially; passing the inner shaft of the delivery system through the central opening of the compressed self-expanding heart valve; attaching the distal tip to the inner shaft of the delivery system; advancing the outer sheath onto the compressed heart valve to prevent radial expansion of at least part of the heart valve; and removing the tubular retainer from the compressed self-expanding heart valve.
15. A catheter system comprising in combination: i. a steering means; and ii. a delivery means for a heart valve, optionally balloon expandable or self-expandable, wherein the steering means and the delivery means are coaxially and rotationally independently movable.
16. Catheter system according to aspect 15, wherein said system is compatible with an introducer for intravascular, optionally transfemoral, applications.
17. Catheter system according to aspect 16, wherein the introducer comprises an introducer sheath which extends or is extendable through the vascular duct and optionally proximal to the target site of the heart.
18. Catheter system according to aspect 15 or 16 having a first and a second retaining mean at the distal end of the catheter system to reversibly secure the heart valve to the delivery means.
19. Catheter system according to aspect 18 wherein the first retaining mean reversibly secures the distal end of the heart valve to the delivery means and the second retaining mean reversibly secures the proximal end of the heart valve to the delivery means.
20. Catheter system according to any of the preceding aspects wherein the heart valve is compressible to the inner diameter of the introducer sheath.
21. Catheter system according to aspect 20, wherein the inner diameter if the introducer sheeth is less than 24 french, preferably less than 20 french, more preferably less than 18 french and even more preferably less than 16 french.
22. Catheter system according to any of the preceding aspects wherein the introducer sheath has a pre-shaped curved configuration.
23. Catheter system wherein the heart valve has male connector elements at the distal and proximal end to engage with female connector elements on the delivery means.
24. Catheter system wherein the delivery means and steering means are operated by one or two means, optionally one or two handles in operative relationship to the pre-shaped introducer sheath.
25. Catheter system according to aspect 15, wherein the steering means have means to deflect the shaft of the delivery catheter from a relaxed configuration into a curved configuration.
26. A delivery system for a heart valve comprising i. a steering means; ii. a delivery means for a heart valve and iii. a introducer sheath wherein the steering means, the delivery means and the introducer sheath are coaxially and circumferentially independently movable.
27. Delivery system according to aspect 28, wherein the introducer sheath extends or is extendable through the vascular duct and optionally proximal to the heart.
28. A method for the delivery of a heart valve to the heart of a patient comprising the steps of: i. in a first step placing an introducer comprising an introducer sheeth into a patient's vasculature; ii. in a second step introducing a catheter system having fixed thereto a self-expanding heart valve through the introducer into the patient's vasculature; iii. in a third step positioning the self-expanding heart valve proximal to the target site in the patient's heart, optionally positioning the heart valve substantially central in the target area; iv. in a fourth step positioning the self-expanding heart valve at the target site; v. in a fifth step partially releasing the self-expanding heart valve from the catheter system; vi. in a sixth step fully releasing the self-expanding heart valve from the catheter system.
29. The method according to aspect 31, wherein the self-expanding heart valve is compressed from a first diameter to a second diameter that is smaller than the first diameter when the self-expanding heart valve is passed through the introducer sheath.
30. A method of loading a self-expanding heart valve onto a delivery means of a catheter system having an inner shaft, an attachable distal tip, and at least one outer sheath, consisting of: compressing the self-expanding heart valve radially and placing the compressed self-expanding heart valve into a tubular retainer, the retainer preventing the self-expanding valve from expanding radially; passing the inner shaft of the delivery system through the central opening of the compressed self-expanding heart valve; attaching the distal tip to the inner shaft of the delivery system; advancing the outer sheath onto the compressed heart valve to prevent radial expansion of at least part of the heart valve; and removing the tubular retainer from the compressed self-expanding heart valve.
31. A system for delivering a self-expanding implant to an implant site within a blood vessel of a patient consisting of an introducer sheath and a delivery catheter, further comprising: the introducer sheath extending from outside of the body of the patient to the vicinity of the implant site; the delivery catheter having a first and a second retaining mean at the distal end of the delivery catheter to reversibly secure the implant to the delivery catheter; the first retaining mean reversibly secures the distal end of the self-expanding implant to the delivery catheter and the second retaining mean reversibly secures the proximal end of the self-expanding implant to the delivery catheter; wherein the delivery catheter is passed through the introducer sheath to deliver the self-expanding implant to the implant site; and the central segment of the self-expanding implant is compressed from a first diameter to a second diameter that is smaller than the first diameter when self-expanding implant is passed through the introducer sheath.
32. The system of aspect 35, wherein the implant is a stent, a stent graft, or a heart valve prosthesis.
33. The system of aspect 35, wherein the implant has male connector elements at the distal and proximal end to engage with female connector elements on the delivery catheter.
34. The system of aspect 37, where the male connector elements are eyelets and the female elements are recesses in the delivery catheter.
35. The system of aspect 35, wherein the introducer sheath has a hemostasis valve at the proximal end.
36. The system of aspect 35, wherein the retaining means consists of axially moveable sheaths that at least partially restrict radial expansion of the implant.
37. A method of delivering a self-expanding implant to an implant site within a blood vessel consisting of: inserting an introducer sheath from outside the body through an opening in the body into a blood vessel; placing the distal end of the introducer sheath in the vicinity of the implant site; reversibly securing the distal end and the proximal end of the self-expanding implant to the distal end of a delivery catheter; inserting the delivery catheter with the self-expanding secured to the delivery catheter into the introducer sheath wherein the central segment of the self-expanding implant is compressed from a first diameter to a second diameter when inserted into the introducer sheath; advancing the delivery catheter through the introducer sheath until the self-expanding implant exits the distal end of the introducer sheath; and releasing the distal end and the proximal end of the self-expanding implant from the delivery catheter at the implant site.
38. A system for delivering an implant to an implant site within a blood vessel of a patient consisting of an introducer sheath, a delivery catheter, and a biasing member, wherein: the introducer sheath extending from outside of the body of the patient to the vicinity of the implant site; the delivery catheter having retaining means to reversibly retain an implant at the distal end of the delivery catheter; the biasing member having means to deflect the shaft of the delivery catheter from a relaxed configuration into a curved configuration; wherein the delivery catheter is passed through the introducer sheath to deliver the implant to the implant site; and, the biasing member is axially and rotationally moveable inside the introducer sheath with respect to the introducer sheath and the delivery catheter.
39. A system of aspect 38, wherein the implant is a self-expanding or balloon expandable heart valve prosthesis.
40. A system of aspect 38, wherein the implant site is the native aortic or mitral valve.
41. A system of aspect 39, wherein the introducer sheath has a pre-shaped curved configuration.
42. A system for maneuvering an implant to an implant site within a blood vessel of a patient consisting of an introducer sheath, a delivery catheter, and a biasing member, whereby: the delivery catheter having retaining means to reversibly retain an implant at the distal end of the delivery catheter; the biasing member having means to deflect the shaft of the delivery catheter from a relaxed configuration into a curved configuration; the delivery catheter is inserted into the introducer sheath and is axially and rotationally moveable within the introducer sheath; and the biasing member is inserted into the introducer sheath and is axially and rotationally moveable within the introducer sheath and is axially and rotationally moveable with respect to the delivery catheter. A method of positioning an implant toward a target site within a blood vessel consisting of inserting an introducer sheath from outside the body through an opening in the body into a blood vessel, wherein emplacement is achieved by: placing the distal end of the introducer sheath in the vicinity of the implant site; reversibly securing the implant to the distal end of a delivery catheter; placing a biasing member having means to actively deflect the shaft of the delivery system from a relaxed configuration into a curved configuration onto the shaft of the delivery system; inserting the delivery catheter with the implant and biasing member into the introducer sheath; and advancing the delivery catheter through the introducer sheath until the implant exits the distal end of the introducer sheath.
43. A method of loading a self-expanding heart valve onto a delivery catheter having an inner shaft, an attachable distal tip, and at least one outer sheath, consisting of: compressing the self-expanding heart valve radially and placing the compressed self-expanding heart valve into a tubular retainer, the retainer preventing the self-expanding valve from expanding radially; passing the inner shaft of the delivery system through the central opening of the compressed self-expanding heart valve; attaching the distal tip to the inner shaft of the delivery system; advancing the outer sheath onto the compressed heart valve to prevent radial expansion of at least part of the heart valve; removing the tubular retainer from the compressed self-expanding heart valve; adjusting the axially and rotationally position of the biasing member within the introducer sheath; activating the biasing member to deflect the shaft of the delivery system into a desired bend configuration; adjusting the axially position of the delivery system to place the implant at the target site; and releasing the implant from the delivery system.
44. Novel enhanced transcatheter heart valve delivery systems, comprising, in combination: at least a specialized prosthetic; a prosthetic delivery system; and at least a valve loader.
45. The system of aspect 44, or any preceding aspects, further comprising a computer interface having a processor.
46. The system of aspect 45, or any preceding aspects, wherein the processor interfaces with imaging and/or sensing modalities.
47. The system of aspect 46, or any preceding aspects, wherein transapical and/or transfemoral delivery systems emplacement is confirmed by the processor, imaging or sensors and a data stream showing the same is captured, stored and/or transmitted wirelessly.

The embodiments as described in this section above can be combined with the embodiments and with each of the features as depicted in the claims and/or as described above herein.

In particular all aspects 1 to 47 as depicted above and the features contained therein can be combined with the disclosure and in particular with each of the features contained in the attached claims forming part of the disclosure of this application.

The following details will describe and illustrate the above listed alternative and/or exemplary embodiments and wherein the below terms and definitions are to be applied to said aspects 1 to 47: the instant disclosure includes an improved bioproshtetic valve which is indicated for patients with high-grade symptomatic aortic valve stenosis and patients with significant aortic valve regurgitation, who require aortic valve replacement, for whom open heart surgery is associated with increased surgical mortality or patients who are otherwise considered inoperable (e.g. porcelain aorta, chest deformation). Examples of the systems for delivery are described below as proprietary disclosures and ostensively patentable subject matters as offered for consideration.

The system consists of three main components: the Valve Prosthesis, the Delivery System and a separate Valve Loader. Each component comes packaged separately.

The valve prosthesis may be constructed from porcine pericardial tissue that is attached to a self-expanding Nitinol stent scaffold using polyester sutures. The scaffold has a crown design that can collapse to fit inside either a transapical or transfemoral catheter delivery device. The valve prosthesis is available in three valve sizes (23 mm, 25 mm and 27 mm) that accommodate native valve annulus diameters ranging from at least about 21 to 27 mm as indicated in Table 2. The valve comes packaged in a polypropylene jar with glutaraldehyde to maintain sterility. The valve is maintained in the deployed dimensional configuration in the jar with the aid of Polyoxymethylene Ring.

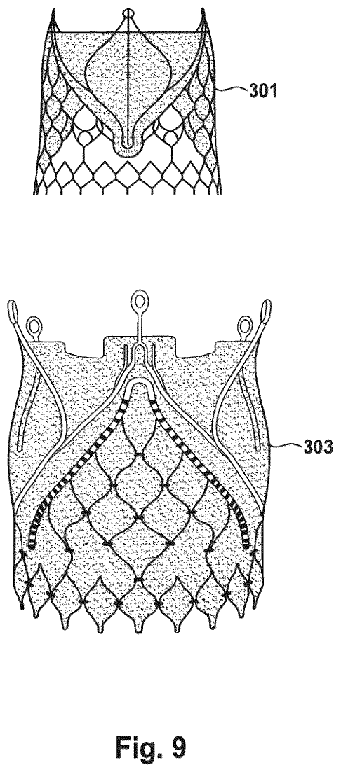

The valve prosthesis may be constructed from porcine pericardial tissue for the leaflets (or cusps) and the skirt. A glutaraldehyde fixation process is used to cross-link and stabilize the collagen structure of the porcine pericardial tissue. The valve leaflets are formed by joining three tissue panels together and attaching them to the metallic stent scaffold using polyester sutures. The prosthesis is sterilized using a solution based sterilization process and then placed in a glutaraldehyde storage solution until ready for implantation. The following sections provide details on each of the structural elements of the prosthesis and a dimensional summary is provided, to demonstrate how the systems of the instant disclosure improve over, and are technically distinct from any known prior art. FIG. 9 shows this device.

Stent Scaffold: The stent scaffold is produced in three different diameters to accommodate the final intended annular diameter of the valve. The nitinol scaffold has a larger diameter than the final dimension of the valve to ensure a constant outward force on the native valve annulus to avoid any valve movement. In addition, the height of the stent scaffold is also increased with increasing valve size. The outward force of the prosthesis, coupled with the unique fixation feature provided by the clipping mechanism of the feelers, mitigates the known risk of valve movement that has been identified with currently marketed TAVR products.

According to the instant disclosure, in embodiments, the nitinol structure is laser cut from a single tube (OD 7 mm and ID 6 mm). The nitinol is etched in a diamond pattern in the “rhombi” section of the scaffold. The stent scaffold consists of three sets of rails that provide attachment for the leaflet side of the pericardial tissue. It also consists of a nitinol structure below the rail termed as the onion region for pericardial skirt tissue attachment. The stent scaffold additionally consists of nitinol support structures termed as feelers that enable the valve to attach directly to the native valve leaflets for implantation. The stent scaffold may contain eyelets on at least on end of the stent that engage with the delivery system to secure the crimped valve to the delivery system.

Pericardial Tissue: The pericardial tissue is obtained from a vendor that supplies porcine tissue for a variety of medical implants. As described above, the pericardial tissue is used for the prosthetic valve leaflets, for the valve skirt that provides a seal onto the native valve and for the feeler cover to protect the native tissue from potential damage. The tissue is attached to the nitinol stent scaffold with standard surgical polyester suture material. A 6-0 suture is used to sew the pericardial material to the commissure tabs and the rhombi of nitinol. A 5-0 suture is used to sew the pericardial material to the rails and of the nitinol scaffold.

Fabric tabs: The commissure posts and feelers each have a PET fabric tab (pledget) sewn in place using standard surgical polyester 6-0 suture material. The fabric tabs at the commissure are in place to provide structural support during valve assembly and the fabric tabs on feelers covering the pericardial tissue are in place to protect the native tissue from potential damage that may be caused by the nitinol metal.

### Valve Implantation Concept

At the outflow section of the valve prosthesis, the self-expanding Nitinol stent scaffold includes three eyelets designed to align with the corresponding recesses at the crown of the delivery system. These mating structures ensure a firm attachment of the prosthesis to the delivery system during the complete course of deployment during implantation. Additional eyelets may be placed on the inflow section of the valve.

According to the instant disclosure, the prosthesis also contains three positional “feelers” that spread apart during delivery of the prosthesis. The feelers are placed in the sinus region behind and at the base of the native leaflets to provide correct positioning with a fixed depth of implant relative to the native leaflet. For enhanced fluoroscopic visualization during the procedure the feelers contain radiopaque markers made of tantalum. The feelers are protected by a small patch of pericardial tissue and fabric sutured to the ends. The Nitinol stent struts at the base of the prosthesis, along with the feelers, provide a securing mechanism of the prosthesis to the native valve leaflets. The inflow of the valve is composed of 24 diamond shaped struts forming a ring that provides the radial strength and seal to securely anchor the valve in position to the native annulus and minimize the paravalvular leakage after implantation.

The rail provides the margin of attachment for the valve leaflets and divides the leaflet and skirt region of the pericardial tissue. The rail is formed in a scalloped shape to mimic the natural shape of a native aortic valve and is designed to allow for commissure deflection which transfers the bulk of the loads from the tissue to the stent to prolong the durability of the prosthesis. The skirt seals the prosthesis to the native annulus.

The instant disclosure contemplates using a transfermoral (TF) delivery system, whereby a prosthetic valve can be inserted via transfemoral delivery using a specially designed catheter system. The approach is to implant the aortic valve prosthesis via transfemoral access using general or local anaesthesia. The TF delivery system is comprised of two catheters: Introducer sheath assembly and a main deployment delivery system.

According to certain embodiments of the instant disclosure, the femoral-iliac vessel will be punctured and the introducer sheath assembly (pre-shaped sheath and introducer) will be inserted into the femoral artery and the tip of the pre-shaped sheath will be positioned in the ascending aorta approximately 2 cm to 4 cm above the native valve. Once in place, the dilator is removed and the pre-shaped sheath is left in position.

The pre-shaped sheath has a generally straight segment to conform to the pathway from the femoral artery to the descending thoracic aorta, and a curved segment to follow the pathway over the arch of the aorta to the aortic valve. The total length of the introducer sheath may be 100 cm to 150 cm. The length of the curved segment may be 15 cm to 30 cm. The radius of curvature of the curved segment may be 3 cm to 15 cm, such as 5 cm to 8 cm. The bend angle from the straight segment to the tip of the sheath maybe 90 degrees to 270 degrees, such as 150 degrees to 210 degrees.

The pre-shaped sheath may be made from flexible polymer material such as PEBAX or Nylon. The wall of the sheath may be reinforced with metal braids or metal coils. The inner wall of the sheath may be lined with PTFE to reduce friction forces. The pre-shaped sheath may have a hydrophilic coating.

A dilator with an atraumatic tip is inserted into the pre-shaped sheath for insertion into the femoral artery and advancement to the aortic valve. The dilator has a guidewire lumen to accommodate a guidewire. The dilator may be pre-shaped. The dilator may be straight. In some embodiments, it may be preferable that the dilator is straight and when inserted into the pre-shaped sheath substantially straightens the curved segment of the pre-shaped sheath for insertion into the femoral artery and passage through the iliac artery and abdominal and descending aorta.

The valve prosthesis is placed into the main delivery system. The inflow segment of the valve prosthesis is restrained by a first sheath. The outflow segment of the valve prosthesis is retrained by a second sheath. The mid-section of the valve prosthesis including the feelers is unconstrained. The first and the second sheath are respectively connected to first and second actuators at the handle of the delivery system. Moving the first actuator distally (toward the tip of the delivery system) moves the first sheath distally and releases the inflow segment of the valve prosthesis. Moving the second actuator proximally (away from the tip of the delivery system) moves the second sheath proximally and releases the outflow segment of the valve prosthesis from the delivery system.

A steerable catheter is mounted on the shaft of the delivery system. The steerable catheter has a handle with a mechanism to deflect the distal segment of the steerable catheter. The mechanism allows for deflection of the steerable catheter by at least 90 degrees from the straight configuration, such as by at least 180 degrees from the straight configuration. Deflecting the steerable catheter will result in a corresponding deflection of the shaft of the delivery system. The steerable catheter is coaxially placed onto the shaft of the delivery system and can move axially and rotationally with respect to the shaft of the delivery system. By rotating the steerable catheter and moving it axially, the location and direction of deflection of the shaft of the delivery system can be selected.

The delivery system with the steerable catheter mounted on the shaft of the delivery system is inserted into the pre-shaped sheath. As the partially crimped valve prosthesis passes into the pre-shaped sheath, the unconstrained mid-section of the valve prosthesis is compressed. The compression of the mid-section of the valve prosthesis may be facilitated by a funnel-shaped inlet into the pre-shaped sheath. Alternatively, a temporary transfer sheath may be placed over the mid-section of the valve prosthesis that compresses the mid-section of the valve prosthesis for insertion into the pre-shaped sheath. The compressed valve prosthesis is advanced through the pre-shaped sheath by pushing the shaft of the delivery system into the pre-shaped sheath.

The distal tip of the pre-shaped sheath is placed in the ascending aorta distal to native aortic valve. When the valve prosthesis exits the distal end of the pre-shaped sheath, the mid-section of the valve prosthesis including the feelers expands. Using the pre-shaped sheath to compress the section of the valve prosthesis for delivery to the native aortic valve, eliminates the need for a separate mechanism to un-sheath the mid-section of the valve prosthesis for release of the feelers.

To place the feelers into the native cusps, it is advantageous to center the valve prosthesis in the aortic root. This can be accomplished by deflecting and rotating the steerable catheter. The point of deflection can be altered by moving the steerable catheter axially along the shaft of the delivery system. Thus, the distal segment of the delivery system can be shaped by the steerable catheter to follow the pathway of the aorta of an individual patient. In some patients the ascending aorta is as short as 3-5 cm. In some patients the ascending aorta is as long as 15-20 cm. In some patients the aortic arch is curved less than 180 degrees. In some patients the aortic arch is curved more than 270 degrees. The degree of deflection of the delivery system can be controlled by the degree of deflection of the steerable catheter. The location of the deflecting region of the delivery system can be controlled by the axial location of the steerable catheter on the delivery system shaft. The direction of deflection of the delivery system can be controlled by the rotational positing of the steerable catheter on the shaft of the delivery system.

An advantage of the pre-shaped sheath is that it deflects the distal end of the delivery system in the general direction of the aortic valve without the use of the steerable catheter. This reduces the bending forces that the steerable catheter has to apply to the shaft of the delivery system to deflect the tip of the delivery system into the final position. Axial movement of the pre-shaped sheath with respect to the delivery system can also be used to adjust the deflection of the shaft of the delivery system. The pre-shaped sheath protects the aortic wall during manipulation of the delivery system and steerable catheter.

Once the distal end of the delivery system is centered in the aortic root with use of the steerable catheter, positioning of the feelers into the native cusps is accomplished by rotationally aligning the feelers with the cusps of the native aortic valve and advancing the valve prosthesis into the aortic annulus. Rotational and axial movement of the valve prosthesis is controlled by axial and rotational movement of the delivery system within the steerable catheter.

Once the feelers are positioned in the native cusps, the valve prosthesis is released by sequentially unsheathing the inflow segment and the outflow segment of the valve prosthesis. Release of the inflow segment of the valve prosthesis anchors the valve prosthesis in the aortic annulus and captures the native leaflets between the feelers and the stent scaffold of the valve prosthesis. Release of the outflow segment of the valve prosthesis disconnects the valve prosthesis from the delivery system.

Alternatively, the outflow segment of the valve prosthesis may be released before the inflow segment of the valve prosthesis. In some cases, it may be preferred to release the inflow segment of the valve prosthesis simultaneously with the outflow segment of the valve prosthesis.

In at least one aspect of the present disclosure, the valve prosthesis may be pre-mounted onto the delivery system by the manufacturer. The mid-section of the valve prosthesis containing the leaflets is unconstrained. The pericardial leaflets located in the mid-section of the valve prosthesis are not compressed avoiding long-term compression damage to the leaflets during shipping and storage. The distal segment including the including the valve prosthesis may be detachable from the shaft of the delivery system. The detached distal segment with the valve prosthesis may be stored separately from the remainder of the delivery system. The distal segment of the delivery system with the mounted partially crimped valve prosthesis may be stored in liquid to avoid de-hydration of the leaflet tissue. Prior to use, the distal segment of the delivery system is connected to the shaft of the delivery system. The connection mechanism used to attach the distal end of the delivery system to the shaft of the delivery system may be in form of male-female thread, magnets, crimping, or bonding.

In another aspect of the present disclosure, the valve prosthesis may have eyelets on at least one end of the stent scaffold to engage the valve prosthesis with the delivery system. The eyelets may sit in recesses in the body of the delivery system. A sheath retains the eyelets in the recesses. By securing the eyelets in the delivery system “melon-seeding” or premature deployment of the self-expanding stent scaffold from the delivery system is prevented. As long as the eyelets are covered by the sheath the valve prosthesis will not deploy. The advantage of this arrangement is that a large segment of the midsection of the valve prosthesis can be un-restrained to allow for full release of the feelers and to minimize compression of the tissue leaflets. Furthermore, the sheaths covering the inflow and outflow segment of the valve prosthesis only require limited travel to release the eyelets in order to disconnect the valve prosthesis from the delivery system.

In another aspect of the present disclosure, the length of the un-restrained midsection of the valve prosthesis is at least 50% of the total length of the valve prosthesis. In another aspect of the present disclosure, the length of the inflow segment of the valve prosthesis restrained by the distal sheath is less than 20% of the length of the valve prosthesis. In another aspect of the present disclosure, the length of the outflow segment of the valve prosthesis restrained by the proximal sheath is less than 20% of the length of the valve prosthesis.

An embodiment of a delivery system according to the present disclosure is represented in FIG. 7. The reference numerals used in FIG. 7 are as follows:

### Main Delivery System Distal Assembly:

| ITEM | COMPONENT | |
|---|---|---|
| NUMBER | DESCRIPTION | MATERIAL |
| 1 | Pre-Shaped | Multi Durometer pebax material (55D + |
| | Introducer | 72D) |
| | Sheath | SS 304 V coil reinforced |
| | | SS 304 V braid reinforced |
| | | PTFE Liner |
| 2 | Steerable Catheter | Multidurometer pebax 72D + 55D + 25D |
| | | 304SS coil, braid wire and pull wire |
| 3 | Stentholder | Multidurometer pebax 55D + Nylon |
| | tubing | Ultem or polyimide stentholder |
| | | PTFE Liner |
| | | 304SS coil, braid wire |
| 4 | Outer Shaft | PTFE Liner |
| | | Multidurometer pebax 72D + 35D |
| | | 304SS braid wire |
| | | Plt/Ir marker band |
| 5 | Guidewire | Pebax, Polyurethane, Polyimide |
| | tubing/Rhombi | |
| | Support | |
| 6 | Rhombi Sleeve | Nylon |
| | | 304SS braid |
| | | Plt/Ir marker band |

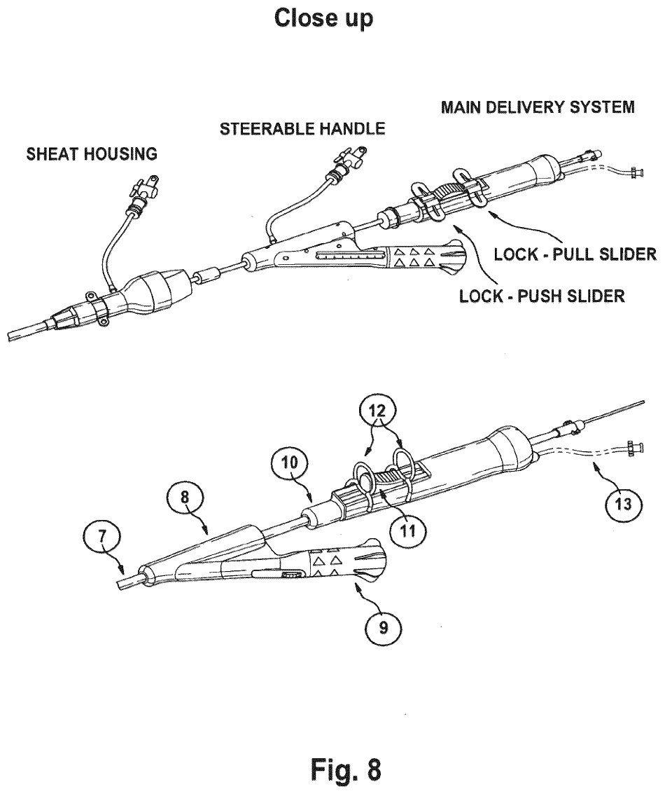

An embodiment of a handle assembly of the delivery system according to the present disclosure is represented in FIG. 8. The reference numerals used in FIG. 8 are as follows:

### Main Delivery System Distal Assembly:

| ITEM | COMPONENT | |
|---|---|---|
| NUMBER | DESCRIPTION | MATERIAL |
| 7 | Shaft of Steerable Sheath | PTFE Liner |
| | | Multidurometer pebax 72D + 35D |
| | | 304SS braid wire |
| | | Plt/Ir marker band |
| | | Stainless steel flat wire |
| 8 | Housing of Steerable | ABS, Polycarbonate |
| | Sheath | |
| 9 | Steerable Rotational | ABS, Polycarbonate, stainless steel |
| | Actuator | |
| 10 | Delivery System Handle | ABS, Polycarbonate |
| 11 | Push-pull Actuator | ABS, Polycarbonate |
| 12 | Safety Locks | Nylon ABS, Polycarbonate |
| 13 | Flush Port | Polyurethane tubing |

The final crimping of the prosthesis is assisted by two loading tubes which allows securing the prosthesis with the outer shaft by distal movement. It is EO-sterilized and for single use only. It should be noted that the TF Valve Loader will utilize the same principle of operation as outlined for the TA Valve Loader.

The present disclosure further relates to a device to facilitate the removal of the distal end of a catheter from an introducer sheath or guiding catheter. Delivery catheters for stents or transcatheter heart valves, for example, may have a split capsule which contains the implant. After the implant is released from the catheter, the open end of the split capsule may be difficult to retract back into the opening of an introducer sheath or guiding catheter within a vessel.

This aspect of the present disclosure provides a tapered insertion device to channel the capsule back into the inner diameter of the introducer sheath or guiding catheter for removal from the body. The device may be static or self-expanding. The device may also include a step to support the implant.

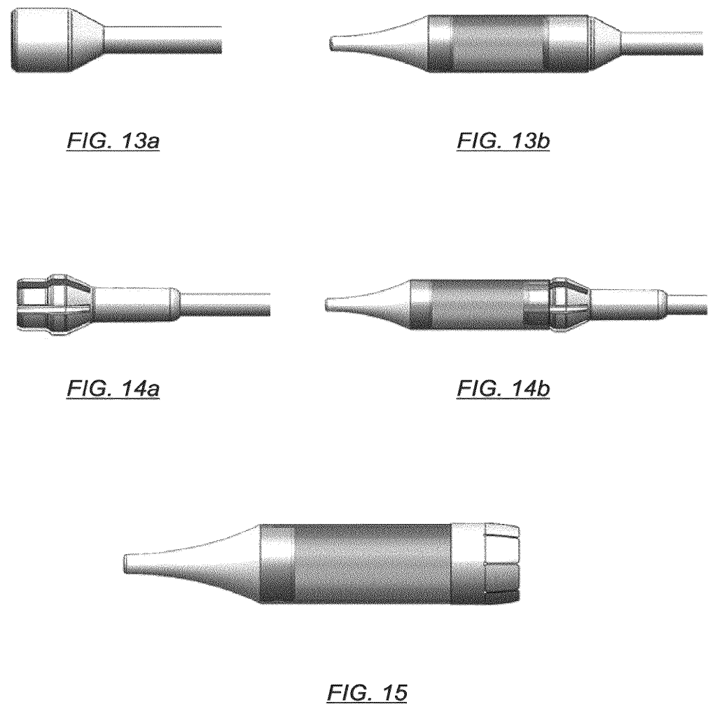

In one embodiment, the device comprises a tapered cylinder that is automatically exposed when capsule is actuated, thereby closing the capsule with a tapered tip. In this regard, reference is made to FIGS. 13a, b. In more detail, FIG. 13a shows one embodiment of the tapered device, and FIG. 13b shows the tapered device with capsule.

In one embodiment (cf. FIGS. 14a, b), the device is self-collapsing, allowing for the device to expand to a larger outer diameter than the capsule when exposed. There is also a straight section on the distal end of the self-collapsing device designed to keep the capsule concentric with the device. The device may be a single piece made from thermoplastic polymer or made from multiple pieces assembled together. In more detail, FIG. 14a shows one embodiment of the self-collapsing device, and FIG. 14b shows the self-collapsing device with capsule.

In another embodiment (cf. FIG. 15), the device is incorporated into the capsule and collapses after implant is released from capsule. The device may be made from a single piece of thermoplastic polymer and may contain slits or living hinges. Collapsing of the device may also be mechanically actuated by the user. In more detail, FIG. 15 shows one embodiment of the collapsing capsule.

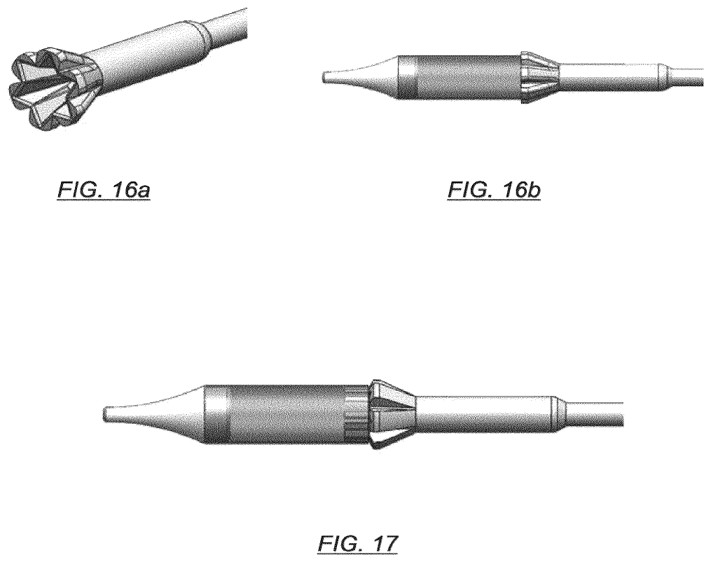

In another embodiment (cf. FIGS. 16a, b), the device is self-expanding. The device is made from thermoplastic polymer with living hinges that allow the device to compress to a smaller diameter that will fit inside the capsule when not in use. When the capsule is opened, the device expands back to its original diameter, larger than the capsule diameter. The self-expanding device is tapered to facilitate entry into the introducer sheath or guiding catheter. FIGS. 16a, b show the device with a step to support the inner diameter of the implant. In more detail, FIG. 16a shows one embodiment of the self-collapsing device, and FIG. 16b shows the self-collapsing device with capsule.

In another embodiment (cf. FIG. 17), the self-expanding device comprises fingers under the capsule to keep the capsule concentric with the device. In more detail, FIG. 17 shows one embodiment of the self-collapsing device with centralizing members.

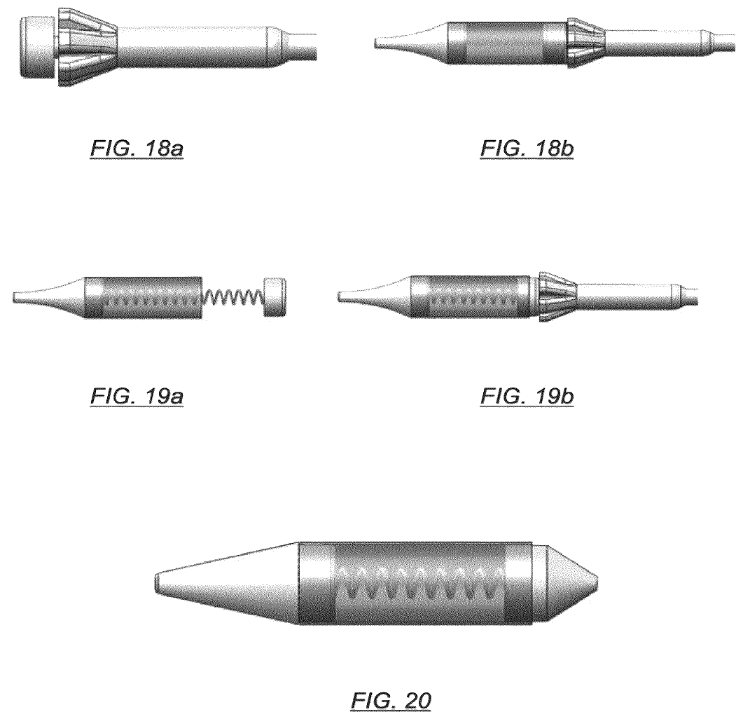

In another embodiment (cf. FIGS. 18a, b), the self-expanding device comprises a stabilizer element distal to the self-expanding wedges. The stabilizer maintains the capsule's concentricity with the device. The profile of the stabilizer may vary. In more detail, FIG. 18a shows one embodiment of the self-expanding device with stabilizer, and FIG. 18b shows the self-expanding device with stabilizer and capsule.

In another embodiment (cf. FIGS. 19a, b), the stabilizer is retractably attached to the inside of the capsule. A spring may be used to actuate the stabilizer. The stabilizer pushes up again the self-expanding device to keep the capsule concentric with the device. The profile of the stabilizer may vary. In more detail, FIG. 19a shows one embodiment of the self-expanding device with stabilizer, and FIG. 19b shows the self-expanding device with stabilizer and capsule.

In another embodiment (cf. FIG. 20), the stabilizer and spring may work without the self-expanding device. The stabilizer may have a tapered or conical shape to facilitate entry into the introducer sheath or guiding catheter. In more detail, FIG. 20 shows one embodiment of the self-actuated device in capsule.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by embodiments of the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the present disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing embodiments of the present disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate embodiments of the present disclosure and does not pose a limitation on the scope of the present disclosure otherwise claimed. No language in the specification should be construed as indicating any non-claimed element essential to the practice of embodiments of the present disclosure.

Groupings of alternative elements or embodiments of the present disclosure described herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out embodiments of the present disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the present disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the present disclosure described herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

### LIST OF REFERENCE NUMBERS

100 delivery means
11 catheter tip
12 catheter shaft
13 handle
14 holding means
15 first retaining means
16 second retaining means
17 first operating means
18 second operating means
19 body of handle
20 first force transmitting means
21 second force transmitting means
22 holding tube
23 catheter end tip
30 steering meens
31 steering catheter tube
32 handle
33 operating means
34 indicator means
35 control wire
36 post section
40 introducer
41 introducer sheath
42 introducer port
43 base member
44 sealing arrangement
45 crimping section

What is claimed:

1. A method for delivering a prosthetic heart valve to a heart of a patient using a catheter system, the method comprising:
inserting an introducer comprising a sheath into the patient;
advancing a delivery catheter and a steering catheter in the introducer, the delivery catheter configured to deliver the prosthetic heart valve and having a proximal end and a distal end, the distal end comprising a tip that includes a first sleeve movable relative to a second sleeve in proximal-distal direction, a middle section configured to be uncovered when the first sleeve is in a proximal-most position with respect to the second sleeve, and a holder disposed in the second sleeve, the second sleeve positioned proximal to the first sleeve and the first sleeve having an open end facing in a proximal direction;
manipulating an integrated handle to cause the steering catheter to deflect to steer the delivery catheter, the integrated handle comprising a delivery handle portion and a steering handle portion comprising a housing, the housing of the steering handle portion sized and configured to receive the delivery handle portion, the integrated handle configured to control movement of the delivery catheter and the steering catheter, independently; and
causing an actuator movably coupled to the delivery handle portion and configured to move with respect to the delivery handle portion to move to manipulate the first sleeve relative to the second sleeve and the holder while the delivery handle portion is received by the housing of the steering handle portion,
wherein the steering catheter is fixedly connected to the steering handle portion and the delivery catheter is fixedly connected to the delivery handle portion, and
wherein the steering catheter and the delivery catheter are selectively coaxially and rotationally independently movable.

2. The method of claim 1, wherein the steering catheter comprises a control wire configured to deflect the steering catheter to steer the delivery catheter.

3. The method of claim 1, wherein the sheath is extendable through a vascular duct and comprises a crimping section.

4. The method of claim 1, wherein the first sleeve is configured to secure a distal end of the prosthetic heart valve to the delivery catheter and the second sleeve is configured to secure a proximal end of the prosthetic heart valve to the delivery catheter, and wherein at least a portion of the prosthetic heart valve is disposed in the middle section of the tip and not covered by the first sleeve or the second sleeve prior to movement of the first sleeve during deployment of the prosthetic heart valve.

5. The method of claim 4, wherein a diameter of the prosthetic heart valve varies over a length of the prosthetic heart valve when the prosthetic heart valve is secured to the delivery catheter.

6. The method of claim 1, wherein an inner diameter of the sheath is less than 24 French.

7. The method of claim 1, wherein the sheath has a pre-shaped, curved configuration.

8. The method of claim 1, wherein the sheath comprises a flexible polymer, a hydrophilic coating, a PTFE liner, coil reinforcement, braid reinforcement, or a combination thereof.

9. The method of claim 1, wherein the delivery catheter comprises a holding tube, and the prosthetic heart valve is secured to the delivery catheter by the holding tube.

10. The method claim 9, wherein a distal end or a proximal end of the prosthetic heart valve comprises a male connector, and the delivery catheter comprises a female connector complimentary to the male connector of the prosthetic heart valve.

11. The method of claim 1, wherein the delivery handle portion comprises a first handle configured to control movement of the delivery catheter and the steering handle portion comprises a second handle configured to control movement of the steering catheter.

12. The method of claim 1, wherein the second handle of the steering catheter comprises a second actuator to deflect a shaft of the delivery catheter from a relaxed configuration into a curved configuration.

13. The method of claim 1, wherein the steering catheter, the delivery catheter, and the sheath are circumferentially independently movable when the steering catheter and the delivery catheter are simultaneously disposed in the sheath.

14. The method of claim 1, wherein the delivery catheter and the steering catheter are simultaneously introducible into the sheath.

15. A method for delivering a prosthetic heart valve to a heart of a patient using a catheter system, the method comprising:
inserting an introducer comprising a sheath and a crimping section proximal to a sheath into the patient, the crimping section having an inner diameter that decreases in a distal direction;
advancing a delivery catheter and a steering catheter in the introducer, the delivery catheter configured to deliver the prosthetic heart valve and having a proximal end and a distal end, the distal end comprising a tip that includes a first sleeve movable relative to a second sleeve in a proximal-distal direction, and a holder disposed in the second sleeve, the second sleeve positioned proximal to the first sleeve and the first sleeve having an open end facing in a proximal direction,
manipulating an integrated handle to cause the steering catheter to deflect to steer the delivery catheter, the integrated handle comprising a delivery handle portion fixedly connected to the delivery catheter and a steering handle portion comprising a housing fixedly connected to the steering catheter, the housing of the steering handle portion sized and configured to receive the delivery handle portion, and the integrated handle configured to control movement of the delivery catheter and the steering catheter, independently; and
causing an actuator movably coupled to the delivery handle portion and configured to move with respect to the delivery handle portion to move to manipulate the first sleeve relative to the second sleeve and the holder while the delivery handle portion is received by the housing of the steering handle portion,
wherein the delivery catheter and the steering catheter are simultaneously introducible into the crimping section of the introducer; and
wherein the steering catheter, the delivery catheter, and the sheath are rotationally independently movable when the steering catheter and the delivery catheter are simultaneously disposed in the sheath.

16. The method of claim 15, wherein a middle section of the tip is uncovered when the first sleeve is in a proximal-most position with respect to the second sleeve.

17. The method of claim 16, wherein the first sleeve secures a distal end of the prosthetic heart valve to the delivery catheter and the second sleeve secures a proximal end of the prosthetic heart valve to the delivery catheter, and wherein at least a portion of the prosthetic heart valve is disposed in the middle section of the tip.

18. The method of claim 17, wherein at least one of the distal end or the proximal end of the prosthetic heart valve comprises a first connector, and the delivery catheter comprises a second connector complimentary to the first connector.

19. The method of claim 18, wherein the first connector comprises eyelets, and the second connector comprises recesses.

20. The method of claim 17, wherein a central segment of the prosthetic heart valve is compressed from a first diameter to a second diameter smaller than the first diameter when the delivery catheter passes through the crimping section.

21. The method of claim 17, wherein a proximal end of the sheath comprises a hemostasis valve.
